(19) United States
(12) Patent Application Publication
(10) Pub. No.: US 2002/0114787 A1
(43) Pub. Date: Aug. 22, 2002

(54) Tolerance to natural antibody antigens

(76) Inventors: John J. Iacomini, Somerville, MA (US); Harout Dersimonian, Wellesley, MA (US); David H. Sachs, Newton, MA (US)

Correspondence Address: HALE AND DORR, LLP, 60 STATE STREET, BOSTON, MA, 02109

(21) Appl. No.: 09/906,387

(22) Filed: Jul. 16, 2001

Related U.S. Application Data
(63) Continuation-in-part of application No. 09/127,027, filed on Jul. 30, 1998, now abandoned.
Application No. 09/127,027 is a continuation-in-part of application No. 08/796,663, filed on Feb. 5, 1997, now abandoned.

(51) Int. Cl. A61K 48/00; C12N 5/06

(52) U.S. Cl. 424/93.21; 435/325

(57) ABSTRACT

Methods of inducing tolerance in a recipient mammal to an antigen or to a graft which expresses the antigen. The methods typically include providing a cell from the recipient mammal which presents the antigen, and allowing the cell to produce or display the moiety in the recipient mammal, thereby inducing tolerance to the antigen.

This application is a continuation-in-part of U.S. application Ser. No. 08/796,663 filed on Feb. 5, 1997, now pending, and of PCT/US98/02141 filed Feb. 5, 1998, pending; both of which are incorporated herein by reference.

The invention relates to the induction of tolerance in graft recipients, particularly xenograft recipients.

### BACKGROUND OF THE INVENTION

Increasing success in organ transplantation has been achieved during the last decade. One consequence of this success is a severe shortage of organ donors; while the number of donors has remained relatively unchanged, the need for organs has continued to rise. Currently, there are more than 33,000 Americans waiting for organ transplants, but only about 4,800 organs donated each year. Because of this growing gap, xenogeneic organ transplantation is an increasingly important area of interest.

Size, availability, and ease of genetic manipulation, have made the pig one of the best studied organ donor species for xenotransplantation. (Sachs, D. H. (1992) “MHC—Homozygous Miniature Swine” in Swine as Models in Biomedical Research, Swindle, M. M. et al. (eds.) (Iowa State University Press, Ames, Iowa, 1992) p.3; Cooper, D. K. C. et al. “The Pig as Potential Organ Donor for Man” in Xenotransplantation, Cooper, D. K. C. et al. (eds.) (Springer-Verlag, Heidelberg, Germany, 1991) p. 481).

Xenogeneic natural antibody-mediated hyperacute rejection is a very significant barrier to xenotransplantation (Platt, J. L and Bach, F. H. (1991) Transplantation 52:937). Overcoming this barrier is important to the long-term success of pig-to-primate xenotransplantation. Recent studies have demonstrated that a predominant epitope on porcine cells recognized by human natural antibodies is a carbohydrate that includes a terminal galactose residue in the conformation of the galactosyl α(1, 3) galactose disaccharide structure (Neethling, F. A. et al. (1994) Transplantation 57:959; Ye, Y. et al. (1994) Transplantation 58:330; Sandrin, M. S. et al. (1993) Proc. Natl. Acad. Sci. USA 90:11391; Good, A. H. et al. (1992) Transplant. Proc. 24:559). Immunopathologic analysis of tissue samples from organs undergoing hyperacute rejection reveals the presence of recipient natural antibodies and complement components along the endothelial surfaces of blood vessels (Leventhal, J. R. et al. (1993) Transplantation 55:857; Leventhal, J. R. et al. (1993) Transplantation 56:1; Platt, J. L. et al. (1991) Transplantation 52:214; Platt, J. L. et al. (1991) Transplantation 52:1037). When recipient natural antibodies are depleted by organ perfusion, hyperacute rejection is delayed or does not occur.

### SUMMARY OF THE INVENTION

The inventors have discovered that an antigen, e.g., a carbohydrate, which reacts with natural antibodies can be used to induce tolerance, in a recipient, to the antigen, thereby inhibiting hyperacute rejection of a graft which includes the antigen. The inhibition of or reduction in natural antibodies which are reactive with the antigen can prolong acceptance, by the recipient, of a graft which includes the antigen, e.g., a carbohydrate antigen.

Accordingly, the invention features, a method of promoting, in a recipient mammal of a first species, tolerance to an antigen, e.g., a carbohydrate moiety, or to a graft which produces or displays the antigen. Preferably, the first species is one which does not produce or display the antigen, e.g., a carbohydrate moiety, on or in its cells, tissues, or organs. By way of example, the recipient mammal can be a human or an Old World primate, e.g., a baboon, (e.g., Papio anubis) or cynomolgus money (Macaca fascicularis).

The method includes:

providing to the recipient mammal a tolerance-inducing antigen, e.g., a carbohydrate moiety, thereby inducing tolerance to the antigen or to a graft which produces or displays the antigen. Although not wishing to be bound by theory, the inventors believe the antigen, e.g., a carbohydrate moiety, mediates the deletion of immune cells which would give rise to antigen-, e.g., carbohydrate moiety-reactive antibodies.

In preferred embodiments the subject is a human and the antigen is one which is not produced or displayed by humans, e.g., a swine antigen.

In preferred embodiments, the antigen, e.g., a carbohydrate moiety, is produced by or displayed on a modified cell of the recipient, wherein the cell has been modified to produce or display the antigen. The cell can be modified in vivo (in the recipient's body), e.g., by in vivo gene therapy or by in vivo treatment with an agent which modifies the cell, or ex vivo (removed from the recipient's body). The cell can be modified by inserting into the cell a nucleic acid which encodes the antigen, (or otherwise promotes the production or display of the antigen) such that the cell produces or displays the antigen. The cell can be modified to produce or display a carbohydrate moiety by inserting into the cell a nucleic acid encoding a protein which promotes, e.g., catalyzes, the formation of the carbohydrate moiety. The encoded protein can be an enzyme which results in the formation of a carbohydrate moiety on the surface of the cell. In particularly preferred embodiments the encoded protein forms the moiety by the addition of a terminal sugar residue to a pre-existing sugar residue on a cell surface molecule.

The cell can be modified to produce or display the antigen, e.g., a carbohydrate moiety, by forming the antigen, e.g., a carbohydrate moiety, on the surface of a cell of the recipient mammal, e.g., by contacting the cell with a protein, e.g., an enzyme, which results in the formation the antigen, e.g., a carbohydrate moiety, on the surface of the cell or by adhering or attaching the antigen to the cell. In particularly preferred embodiments the protein forms the moiety by the addition of a terminal sugar residue to a pre-existing sugar residue on a cell surface molecule.

In preferred embodiments, the method further includes: introducing a graft from a donor mammal into the recipient mammal. The donor can be, for example, a species which normally produces or displays the antigen, e.g., a carbohydrate moiety, on its cells, tissues, or organs. By way of example the donor can be a swine, e.g., a miniature swine, or a New World primate, e.g., a squirrel monkey (Saimiri sciureus). Preferably, the graft expresses a major histocompatibility complex (MHC) antigen. The graft can be an organ, e.g., a heart, liver, or kidney, or skin, or a preparation of hematopoietic stem cells, e.g., a bone marrow preparation. In particularly preferred embodiments the recipient is a human and the graft is from a swine, e.g., a miniature swine.

In preferred embodiments the cell is removed from the recipient, modified so as to allow it to produce or display the antigen, e.g., a carbohydrate, and implanted in the recipient.

In preferred embodiments, the method includes: preferably prior to providing the tolerance-inducing antigen, e.g., a carbohydrate, inactivating immune system cells, e.g., xenoreactive immune cells, e.g, carbohydrate moiety-reactive immune cells, of the recipient.

In preferred embodiments, the method includes: preferably prior to providing the tolerance-inducing antigen, e.g., a carbohydrate, inactivating antibodies, e.g., xenoreactive antibodies, e.g, carbohydrate moiety-reactive antibodies, of the recipient.

In preferred embodiments the method inhibits hyperacute rejection

In preferred embodiments the method further includes providing to the recipient, and inducing tolerance to, a second antigen, e.g., a carbohydrate moiety. The second antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In another aspect, the invention features, a method of promoting, in a recipient mammal of a first species, tolerance to the galactosyl α(1, 3) galactose moiety, or to a graft which produces or displays the galactosyl α(1, 3) galactose moiety. Preferably, the first species is one which does not possess UDP galactose:β-D-galactosyl-1,4-N-acetyl-D-glucosaminide α(1,3)galactosyltransferase (α1,3GT) activity or which does not produce or display galactosyl α(1, 3) galactose moieties on its cells, tissues, or organs. By way of example, the recipient mammal can be a human or an Old World primate, e.g., a baboon, (e.g., Papio anubis) or cynomolgus money (Macaca fascicularis).

The method includes:

providing to the recipient mammal a tolerance-inducing galactosyl α(1, 3) galactose moiety thereby inducing tolerance to the galactosyl α(1, 3) galactose moiety or to a graft which produces or displays the galactosyl α(1, 3) galactose moiety. Although not wishing to be bound by theory, the inventors believe the galactosyl α(1, 3) galactose moiety mediates the deletion of immune cells which would give rise to galactosyl α(1, 3) galactose moiety-reactive antibodies,

In preferred embodiments the galactosyl α(1, 3) galactose moiety is produced or displayed on a modified cell of the recipient, wherein modified means the cell has been modified to produce or display the galactosyl α(1, 3) galactose moiety. The modification can be performed in vivo but is preferably performed ex vivo.

In preferred embodiments, the method further includes: introducing a graft from a donor mammal into the recipient mammal. The donor can be, for example, a species which normally produces or displays the galactosyl α(1, 3) galactose moiety, on its cells, tissues, or organs. By way of example the donor can be a swine, e.g., a miniature swine, or a New World primate, e.g., a squirrel monkey (Saimiri sciureus). Preferably, the graft expresses a major histocompatibility complex (MHC) antigen. The graft can be an organ, e.g., a heart, liver, or kidney, or skin, or a preparation of hematopoietic stem cells, e.g., a bone marrow preparation. In particularly preferred embodiments the recipient is a human and the graft is from a swine, e.g., a miniature swine.

In preferred embodiments, the method includes: inactivating immune system cells, e.g., xenoreactive immune cells, e.g, galactosyl α(1, 3) galactose moiety-reactive immune cells, of the recipient, preferably prior to providing the tolerance-inducing galactosyl α(1, 3) galactose moiety.

In preferred embodiments, the method includes: inactivating antibodies, e.g., xenoreactive antibodies, e.g, carbohydrate moiety-reactive antibodies, of the recipient, preferably prior to providing the tolerance-inducing antigen, e.g., a carbohydrate.

In preferred embodiments the method inhibits hyperacute rejection.

In preferred embodiments the method further includes providing to the recipient, and inducing tolerance to, a second antigen, e.g., a carbohydrate moiety. The second antigen can be produced by or displayed on a modified cell of the recipient. The modified cell can be the same cell which produces or displays the galactosyl α(1, 3) galactose moiety or it can be a different cell. Generally, methods described herein for providing antigen to the recipient can be used to provide the second antigen to the recipient.

In another aspect, the invention features, a method of promoting, in a recipient mammal of a first species, tolerance to the galactosyl α(1, 3) galactose moiety or to a graft which produces or displays the galactosyl α(1, 3) galactose moiety by providing a cell from the recipient mammal which produces or displays the galactosyl α(1, 3) galactose moiety. Preferably, the first species is one which does not possess UDP galactose:β-D-galactosyl-1,4-N-acetyl-D-glucosaminide α(1,3)galactosyltransferase (α1,3GT) activity or which does not produce or display galactosyl α(1,3) galactose moieties on its cells, tissues, or organs, and can be, by way of example, a human or an Old World primate, e.g., a baboon, (e.g., Papio anubis) or cynomolgus money (Macaca fascicularis).

The method includes:

providing a cell from the recipient mammal which produces or displays the galactosyl α(1, 3) galactose moiety (wherein the cell has been modified to produce or display a α(1, 3) galactose moiety); and

preferably, allowing the recipient mammalian cell to produce or display the galactosyl α(1, 3) galactose moiety in the recipient mammal, thereby inducing tolerance to the galactosyl α(1, 3) galactose moiety or to a graft which includes the galactosyl α(1, 3) galactose moiety.

The modification can be performed in vivo but is preferably performed ex vivo.

In preferred embodiments: the cell is modified to produce or display the galactosyl α(1, 3) galactose moiety by inserting into the cell a nucleic acid encoding a protein which promotes, e.g., catalyzes, the formation of the galactosyl α(1, 3) galactose moiety.

In preferred embodiments: the cell is modified to produce or display the galactosyl α(1, 3) galactose moiety by forming the galactosyl α(1, 3) galactose moiety on the surface of a cell of the recipient mammal, e.g., by contacting the cell with a protein, e.g., an enzyme which results in the formation an galactosyl α(1,3) galactose moiety on the surface of the cell. In particularly preferred embodiments the moiety is formed by the addition of a terminal galactosyl residue to a galactosyl residue, e.g., to a galactosyl residue linked to N-acetylglucosaminyl residue, on the surface of the recipient cell, by contacting the cell with an α(1,3)galactosyltransferase, e.g., β-D-galactosyl-1,4-N-acetyl-D-glucosaminide α(1,3)galactosyltransferase.

In preferred embodiments, the method further includes: introducing a graft from a donor mammal into the recipient mammal. The donor can be, for example, a species which normally produces or displays the galactosyl α(1, 3) galactose moiety, on its cells, tissues, or organs. By way of example the donor can be a swine, e.g., a miniature swine, or a New World primate, e.g., a squirrel monkey (Saimiri sciureus). Preferably, the graft expresses a major histocompatibility complex (MHC) antigen. The graft can be an organ, e.g., a heart, liver, or kidney, or skin, or a preparation of hematopoietic stem cells, e.g., a bone marrow preparation. In particularly preferred embodiments the recipient is a human and the graft is from a swine, e.g., a miniature swine.

In preferred embodiments, the method includes: inactivating immune system cells, e.g., xenoreactive immune cells, e.g, galactosyl α(1, 3) galactose moiety-reactive immune cells, of the recipient, preferably prior to providing the recipient cell which produce or displays the galactosyl α(1, 3) galactose moiety.

In preferred embodiments, the method includes: inactivating antibodies, e.g., xenoreactive antibodies, e.g, galactosyl α(1, 3) galactose-reactive antibodies, of the recipient, preferably prior to providing the recipient cell which produce or displays the galactosyl α(1, 3) galactose moiety.

In preferred embodiments the method inhibits hyperacute rejection.

In preferred embodiments the method further includes providing to the recipient, and inducing tolerance to, a second antigen, e.g., a carbohydrate moiety. The antigen can be produced by or displayed on a modified cell of the recipient. The modified cell can be the same cell which produces or displays the galactosyl α(1,3) galactose moiety or it can be a different cell. Generally, methods described herein for providing antigen to the recipient can be used to provide the second antigen to the recipient.

In another aspect, the invention features, a method of promoting, in a recipient mammal of a first species, tolerance to the galactosyl α(1, 3) galactose moiety or to a graft which produces or displays the galactosyl α(1, 3) galactose moiety by providing a cell from the recipient mammal, into which cell has been inserted a nucleic acid encoding a protein which promotes, e.g., catalyzes, the formation of the galactosyl α(1, 3) galactose moiety. Preferably, the first species is one which does not possess UDP galactose:β-D-galactosyl-1,4-N-acetyl-D-glucosaminide α(1,3)galactosyltransferase (α1,3GT) activity or which does not produce or display galactosyl α(1, 3) galactose moieties on its cells, tissues, or organs, and can be, by way of example, a human or an Old World primate, e.g., a baboon, (e.g., Papio anubis) or cynomolgus money (Macaca fascicularis).

The method includes:

providing a cell from the recipient mammal, into which cell has been inserted a nucleic acid encoding a protein which promotes, e.g., catalyzes, the formation of the galactosyl α(1, 3) galactose moiety; and

preferably, allowing the recipient mammalian cell to produce or display the galactosyl ((1, 3) galactose moiety in the recipient mammal, thereby inducing tolerance to the galactosyl α(1, 3) galactose moiety or to a graft which produce or displays the galactosyl α(1, 3) galactose moiety.

Insertion of the nucleic acid can be done in vivo but is preferably done ex vivo.

In preferred embodiments: the nucleic acid encodes a protein which promotes the addition of a terminal galactosyl residue to a galactosyl residue, e.g., to a galactosyl residue linked to N-acetylglucosaminyl residue; the nucleic acid encodes a mammalian, e.g., a vertebrate, e.g., porcine or murine α(1,3)galactosyltransferase; the nucleic acid encodes a New World primate, e.g., a squirrel monkey, α(1,3)galactosyltransferase; the nucleic acid encodes an α(1,3)galactosyltransferase, e.g., UDP galactose:β-D-galactosyl-1,4-N-acetyl-D-glucosaminide α(1,3)galactosyltransferase.

In preferred embodiments, the method further includes: introducing a graft from a donor mammal into the recipient mammal. The donor can be, for example, a species which normally produces or displays the galactosyl α(1, 3) galactose moiety on its cells, tissues, or organs, or a species which possesses a(1,3)galactosyltransferase activity. By way of example the donor can be a swine, e.g., a miniature swine, or a New World primate, e.g., a squirrel monkey (Saimiri sciureus). Preferably, the graft expresses a major histocompatibility complex (MHC) antigen. The graft can be an organ, e.g., a heart, liver, or kidney, or skin, or a preparation of hematopoietic stem cells, e.g., a bone marrow preparation. In particularly preferred embodiments the recipient is a human and the graft is from a swine, e.g., a miniature swine.

The recipient cell can be any cell suitable for production or display of the galactosyl α(1, 3) galactose moiety, e.g., a hematopoietic cell. Hematopoietic stem cells, e.g., bone marrow cells, which are capable of developing into mature myeloid and/or lymphoid cells, are particularly preferred. It is possible that later stage cells can be used, since the transgene(α(1,3)galactosyltransferase) should modify endogenous proteins causing them to be recognized as self. Stem cells derived from the cord blood of the recipient can be used in methods of the invention. Other cells suitable for use in the invention include peripheral blood cells. Suitable cells are those which can produce or display the galactosyl α(1, 3) galactose moiety and tolerize the animal. Although not wishing to be bound by theory, the inventors believe that suitable recipient cells are cells which produce or display the galactosyl α(1, 3) galactose moiety such that the moiety can interact with immune cells at an early stage in their development. Although not wishing to be bound by theory, this is believed to allow deletion of cells which would give rise to galactosyl α(1, 3) galactose moiety reactive antibodies. Suitable cells are those which result in tolerance as opposed to an immune response.

In other preferred embodiments, the providing step of the method includes: removing the recipient mammalian cell from the recipient mammal prior to introducing the nucleic acid into the recipient mammal cell and administering the recipient mammalian cell to the recipient mammal.

In preferred embodiments, the method includes: inactivating immune system cells, e.g., xenoreactive immune cells, e.g, galactosyl α(1, 3) galactose moiety-reactive immune cells, of the recipient, preferably prior to providing the recipient cell.

In preferred embodiments, the method includes: inactivating antibodies, e.g., xenoreactive antibodies, e.g, galactosyl α(1, 3) galactose-reactive antibodies, of the recipient, preferably prior to providing the recipient cell.

In preferred embodiments, the method includes an additional step which inactivates a recipient anti-galactosyl α(1, 3) galactose antibody. For example, anti-galactosyl (α1, 3) galactose epitope antibody activity can be inactivated prior to the introduction or formation in the recipient of a recipient cell which produce or displays galactosyl α(1, 3) galactose moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-galactosyl α(1, 3) galactose antibody; depleting natural antibodies from the blood of the recipient, e.g., by hemoperfusing an organ, e.g., a liver or kidney, obtained from a mammal of the donor species or by contacting the blood of the recipient with galactosyl α(1, 3) galactose moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method inhibits hyperacute rejection.

In preferred embodiments the method further includes providing to the recipient, and inducing tolerance to, a second antigen, e.g., a carbohydrate moiety. The second antigen can be produced by or displayed on a modified cell of the recipient. The modified cell can be the same cell which produces or displays the galactosyl α(1,3) galactose moiety or it can be a different cell. Generally, methods described herein for providing antigen to the recipient can be used to provide the second antigen to the recipient.

In another aspect, the invention features, a method of promoting, in a recipient mammal of a first species, tolerance to the galactosyl α(1, 3) galactose moiety or to a graft which produce or displays the galactosyl α(1, 3) galactose moiety by forming the galactosyl α(1, 3) galactose moiety on the surface of a cell of the recipient mammal. Preferably, the first species is one which does not possess UDP galactose:β-D-galactosyl-1,4-N-acetyl-D-glucosaminide α(1,3)galactosyltransferase (α1,3GT) activity or which does not produce or display galactosyl α(1, 3) galactose moieties on its cells, tissues, or organs and can be, by way of example, a human or an Old World primate, e.g., a baboon, (e.g., Papio anubis) or cynomolgus money (Macaca fascicularis).

The method includes:

forming the galactosyl α(1, 3) galactose moiety on the surface of a cell of the recipient mammal;

preferably, allowing the recipient mammalian cell to produce or display the galactosyl α(1, 3) galactose moietv in the recipient mammal, thereby inducing tolerance to the galactosyl α(1, 3) galactose moiety.

The formation can be effected in vivo but is preferably effected ex vivo.

In preferred embodiments the galactosyl α(1,3) galactose moiety is formed by contacting the cell with a protein, e.g., an enzyme which results in the formation an galactosyl α(1,3) galactose moiety on the surface of the cell.

In preferred embodiments the moiety is formed by the addition of a terminal galactosyl residue to a galactosyl residue, e.g., to a galactosyl residue linked to N-acetylglucosaminyl residue, on the surface of the recipient cell. Addition of the terminal residue can be promoted by contacting the recipient cell with a protein which promotes the addition of a terminal galactosyl residue. By way of example, the protein can be: a protein which promotes the addition of a terminal galactosyl residue to a galactosyl residue, e.g., to a galactosyl residue linked to N-acetylglucosaminyl residue; a mammalian, e.g., a vertebrate, e.g., porcine or murine α(1,3)galactosyltransferase; a New World primate, e.g., a squirrel monkey, α(1,3)galactosyltransferase; an α(1,3)galactosyltransferase, e.g., UDP galactose:β-D-galactosyl-1,4-N-acetyl-D-glucosaminide α(1,3)galactosyltransferase.

In preferred embodiments, the method further includes: introducing a graft from a donor mammal into the recipient mammal. The donor can be, for example, a species which normally produces or displays the galactosyl α(1, 3) galactose moiety on its cells, tissues, or organs, or a species which possesses α(1,3)galactosyltransferase activity. By way of example the donor can be a swine, e.g., a miniature swine, or a New World primate, e.g., a squirrel monkey (Saimiri sciureus). Preferably, the graft expresses a major histocompatibility complex (MHC) antigen. The graft can be an organ, e.g., a heart, liver, or kidney, or skin, or a preparation of hematopoietic stem cells, e.g., a bone marrow preparation. In particularly preferred embodiments the recipient is a human and the graft is from a swine, e.g., a miniature swine.

In preferred embodiments, the method includes: inactivating immune system cells, e.g., xenoreactive immune cells, e.g, galactosyl α(1, 3) galactose moiety-reactive immune cells, of the recipient, preferably prior to providing the recipient cell.

In preferred embodiments, the method includes: inactivating an antibodies, e.g., xenoreactive antibodies, e.g, galactosyl α(1, 3) galactose-reactive antibodies, of the recipient, preferably prior to providing the recipient cell.

In preferred embodiments, the method includes an additional step which inactivates a recipient anti-galactosyl α(1, 3) galactose antibody. For example, anti-galactosyl α(1, 3) galactose antibody activity can be inactivated prior to the introduction or formation in the recipient of a recipient cell which produce or displays galactosyl α(1, 3) galactose moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-galactosyl α(1, 3) galactose epitope antibody; depleting natural antibodies from the blood of the recipient, e.g., by hemoperfusing an organ, e.g., a liver or kidney, obtained from a mammal of the donor species or by contacting the blood of the recipient with galactosyl α(1, 3) galactose moieties coupled to an insoluble substrate; administering to the recipient drugs which inactivate natural antibodies, e.g., deoxyspergualin (DSG) (Bristol-Myers Squibb Co., Princeton, N.J.); or administering to the recipient anti-IgM antibodies.

In preferred embodiments the method inhibits hyperacute rejection.

In preferred embodiments the method further includes providing to the recipient, and inducing tolerance to, a second antigen, e.g., a carbohydrate moiety. The second antigen can be produced by or displayed on a modified cell of the recipient. The modified cell can be the same cell which produces or displays the galactosyl α(1, 3) galactose moiety or it can be a different cell. Generally, methods described herein for providing antigen to the recipient can be used to provide the second antigen to the recipient.

In another aspect, the invention features, a method of promoting, in a recipient mammal, e.g., a human, tolerance to an antigen, e.g., a carbohydrate moiety, e.g., a blood group carbohydrate, from a donor mammal of the same species, wherein the antigen is not expressed in the recipient but is expressed in the donor.

The method includes:

providing to the recipient mammal a tolerance-inducing antigen, e.g., a carbohydrate moiety, e.g., a blood group carbohydrate, thereby inducing tolerance to the antigen or to a graft which produces or displays the antigen. Although not wishing to be bound by theory, the inventors believe the antigen, e.g., a carbohydrate moiety, mediates the deletion of immune cells which would give rise to antigen-, e.g., carbohydrate moiety-reactive antibodies.

The donor can be, for example, an animal which has or expresses an allele which results in production or display of the antigen and the recipient can be an animal that lacks, or fails to express, an allele which results in production or display of the antigen. For example, the antigen can be an antigen which conditions blood type. Human blood group carbohydrate epitopes are found at the nonreducing termini of protein- and lipid bound oligosaccharides. The genes for numerous enzymes which synthesize blood group antigen determinants have been cloned. These enzymes act on the Galβ1,3/4GIcNAc moieties of N- and O-glycans and glycolipids. The carbohydrate groups which characterize the various blood groups are referred to herein as blood group antigens, carbohydrates, or moieties. The human blood group A, B, H, Le and I epitopes are synthesized, respectively, by UDP-GalNAc:Fucα1,2Gal-R α1,3-GalNAc transferase (EC 2.4.1.40), UDP-GalNAc:Fucα1,2Gal-Rα1,3Gal transferase (EC 2.4.1.37), GDP-Fuc:βgalactosideα2-Fuc-transferase (EC 2.4.1.69), GDP-Fuc:Galβ1,3/4GlcNAc-Rα4/3Fuc transferase (EC 2.4.1.65), and UDP-GlcNAc:GlcNAcβ1,3Galβ1,4GlcNAc-Rβ6-GlcNAc transferase.

In preferred embodiments the subject is a human and the antigen is a blood group A carbohydrate moiety.

In preferred embodiments the subject is a human and the antigen is a blood group B carbohydrate moiety.

In preferred embodiments the subject is a human and the antigen is a blood group H carbohydrate moiety.

In preferred embodiments the subject is a human and the antigen is a blood group Le carbohydrate moiety.

In preferred embodiments the subject is a human and the antigen is a blood group I carbohydrate moiety.

In preferred embodiments a recipient cell is modified to express UDP-GalNAc:Fucα1,2Gal-R α1,3-GalNAc transferase (EC 2.4.1.40), or an enzyme of equivalent activity, e.g., by insertion of nucleic acid which encodes the enzyme into a cell of the recipient.

In preferred embodiments a recipient cell is modified to express UDP-GalNAc:Fucα1,2Gal-Rα1,3Gal transferase (EC 2.4.1.37), or an enzyme of equivalent activity, e.g., by insertion of nucleic acid which encodes the enzyme into a cell of the recipient.

In preferred embodiments a recipient cell is modified to express GDP-Fuc:βgalactosideα2-Fuc-transferase (EC 2.4.1.69), or an enzyme of equivalent activity, e.g., by insertion of nucleic acid which encodes the enzyme into a cell of the recipient.

In preferred embodiments a recipient cell is modified to express GDP-Fuc:Galβ1,3/4GlcNAc-Rα4/3Fuc transferase (EC 2.4.1.65), or an enzyme of equivalent activity, e.g., by insertion of nucleic acid which encodes the enzyme into a cell of the recipient.

In preferred embodiments a recipient cell is modified to express UDP-GlcNAc:GlcNAcβ1,3Galβ1,4GlcNAc-Rβ6-GlcNAc transferase, or an enzyme of equivalent activity, e.g., by insertion of nucleic acid which encodes the enzyme into a cell of the recipient.

In preferred embodiments, the antigen, e.g., a carbohydrate moiety, is produced by or displayed on a modified cell of the recipient, wherein modified means the cell has been modified to produce or display the antigen. The cell can be modified in vivo (in the recipient's body), e.g., by in vivo gene therapy or by in vivo treatment with an agent which modifies the cell, or ex vivo (removed from the recipient's body) by recombinant means or by treatment with an agent which modifies the cell. The cell can be modified to produce or display an antigen by inserting into the cell a nucleic acid encoding the antigen or nucleic acid encoding a protein (or proteins) which promotes, e.g., catalyzes, the formation of the antigen, e.g., a carbohydrate moiety.

The encoded protein can be an enzyme, e.g., a transferase, which results in the formation an carbohydrate moiety on the surface of the cell. E.g., the cell can be modified to express an enzyme (or enzymes) which promotes the formation of a blood group carbohydrate moiety or moieties not produced or displayed by the recipient, e.g., one or more of UDP-GalNAc:Fucα1,2Gal-R α1,3-GalNAc transferase (EC 2.4.1.40), UDP-GalNAc:Fucα1,2Gal-Rα1,3Gal transferase (EC 2.4.1.37), GDP-Fuc:βgalactoside α2-Fuc-transferase (EC 2.4.1.69), GDP-Fuc:Galβ1,3/4GlcNAc-Rα4/3Fuc transferase (EC 2.4.1.65), and UDP-GlcNAc:GlcNAcβ1,3Galβ1,4GlcNAc-Rβ6-GlcNAc transferase. In particularly preferred embodiments the encoded protein forms the moiety by the addition of one or more terminal sugar residues to a pre-existing sugar on a cell surface molecule.

The cell can be modified to produce or display the antigen, e.g., a carbohydrate moiety, by forming the antigen, e.g., a carbohydrate moiety, on the surface of a cell of the recipient mammal, e.g., by contacting the cell with a protein, e.g., an enzyme, which results in the formation, e.g., by attachment, of the antigen, e.g., a carbohydrate moiety, on the surface of the cell. In particularly preferred embodiments the protein forms the moiety by the addition of one or more terminal sugar residues to a pre-existing sugar residue on a cell surface molecule.

In preferred embodiments, the method further includes: introducing a graft from a donor mammal into the recipient mammal.

In preferred embodiments the cell is removed from the recipient, modified so as to allow it to produce or display the antigen, e.g., a carbohydrate, and implanted in the recipient.

In preferred embodiments, the method includes: preferably prior to providing the tolerance-inducing antigen, e.g., a carbohydrate, inactivating immune system cells, e.g., antigen-reactive immune cells, e.g, carbohydrate moiety-reactive immune cells, of the recipient.

In preferred embodiments the method inhibits hyperacute rejection. In preferred embodiments, the method includes: preferably prior to providing the tolerance-inducing antigen, e.g., a carbohydrate, inactivating antibodies, e.g., antigen-reactive antibodies, e.g, carbohydrate moiety-reactive antibodies, of the recipient.

In preferred embodiments the method further includes providing to the recipient, and inducing tolerance to, a second antigen, e.g., a carbohydrate moiety. The second antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments, the method further includes: introducing a graft from a donor mammal into the recipient mammal.

In another aspect, the invention features, a method of promoting, in a recipient mammal, e.g., a human, tolerance to a blood group A carbohydrate antigen, e.g., a terminal N-acetyl-D-galactosamine moiety, or to a graft which produces or displays a blood group A carbohydrate, e.g., a terminal N-acetyl-D-galactosamine moiety. The blood group sugar can be Type 1 or Type 2. Preferably, the recipient does not possess an enzyme which promotes the formation of a blood group A carbohydrate, e.g., UDP-GalNAc:Fucα1,2Gal-Rα1,3-GalNAc transferase (EC 2.4.1.40)), or an enzyme of equivalent activity, or does not produce or display a group A carbohydrate, e.g., a terminal N-acetyl-D-galactosamine moiety on its cells, tissues, or organs.

The method includes:

providing to the recipient mammal a tolerance-inducing blood group A carbohydrate antigen, e.g., a terminal N-acetyl-D-galactosamine moiety, thereby inducing tolerance to the blood group A moiety or to a graft which produce or displays that moiety. Although not wishing to be bound by theory, the inventors believe the carbohydrate moiety mediates the deletion of immune cells which would give rise to blood group A-reactive antibodies.

The donor can be, for example, an animal which has or expresses an allele which results in production or display of the antigen and the recipient can be an animal that lacks, or fails to express, an allele which results in production or display of the antigen.

In preferred embodiments a blood group A carbohydrate moiety is produced or displayed on a modified cell of the recipient, wherein the cell has been modified to produce or display the blood group A carbohydrate moiety. The cell can be modified in vivo or ex vivo.

In preferred embodiments: the cell is modified to produce or display a blood group A carbohydrate moiety by inserting into the cell a nucleic acid which encodes a protein which promotes, e.g., catalyzes, the formation of the blood group A carbohydrate moiety, e.g., UDP-GalNAc:Fucα1,2Gal-Rα1,3-GalNAc transferase (EC 2.4.1.40) or an enzyme with equivalent activity.

In other preferred embodiments, the providing step of the method includes: removing the recipient mammalian cell from the recipient mammal prior to introducing nucleic acid into the recipient mammal cell and administering the recipient mammalian cell to the recipient mammal.

In preferred embodiments: the cell is modified to produce or display a blood group A carbohydrate moiety by forming the blood group A carbohydrate moiety on the surface of a cell of the recipient mammal, e.g., by contacting the cell with a protein, e.g., an enzyme which promotes the formation of the blood group A carbohydrate moiety on the surface of the cell. In particularly preferred embodiments the moiety is formed by the addition of a blood group A carbohydrate, on the surface of the recipient cell, by contacting the cell with an enzyme which promotes the synthesis or attachment of the moiety, e.g., UDP-GalNAc:Fucα1,2Gal-Rα1,3-GalNAc transferase (EC 2.4.1.40) or an enzyme with equivalent activity.

In preferred embodiments, the method includes inactivating immune system cells, e.g., antigen-reactive immune cells, e.g, blood group A carbohydrate moiety-reactive immune cells, of the recipient, preferably prior to providing the recipient cell which produces or displays a blood group A carbohydrate moiety.

In preferred embodiments, the method includes: inactivating antibodies, e.g., antigen-reactive antibodies, e.g, blood group A carbohydrate-reactive antibodies, of the recipient, preferably prior to providing the recipient cell which produces or displays a blood group A carbohydrate moiety.

In preferred embodiments the method inhibits hyperacute rejection.

The recipient cell can be any cell suitable for presentation of a blood group A carbohydrate moiety, e.g., a hematopoietic cell. Hematopoietic stem cells, e.g., bone marrow cells, which are capable of developing into mature myeloid and/or lymphoid cells, are particularly preferred. It is possible that later stage cells can be used. Stem cells derived from the cord blood of the recipient can be used in methods of the invention. Other cells suitable for use in the invention include peripheral blood cells. Suitable cells are those which can produce or display the blood group carbohydrate moiety and tolerize the animal. Although not wishing to be bound by theory, the inventors believe that suitable recipient cells are cells which produce or display the blood group carbohydrate moiety such that the moiety can interact with immune cells at an early stage in their development. Although not wishing to be bound by theory, this is believed to allow deletion of cells which would give rise to blood group A reactive antibodies. Suitable cells are those which result in tolerance as opposed to an immune response.

In preferred embodiments, the method includes an additional step which inactivates a recipient anti-blood group A carbohydrate antibody. For example, anti-blood group A carbohydrate antibody activity can be inactivated prior to the introduction or formation in the recipient of a recipient cell which produces or displays group A carbohydrate moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-group A carbohydrate antibody; depleting natural antibodies from the blood of the recipient, e.g., by hemoperfusing an organ, e.g., a liver or kidney, obtained from a mammal of the donor species or by contacting the blood of the recipient with blood group A moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method further includes providing to the recipient, and inducing tolerance to, a second antigen. The antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments more than one blood group carbohydrate moiety is provided to the recipient. The second carbohydrate moiety can be provided by a method described herein.

In preferred embodiments, the method further includes: introducing a graft from a donor mammal into the recipient mammal.

In another aspect, the invention features, a method of promoting, in a recipient mammal, e.g., a human, tolerance to a blood group B carbohydrate antigen or to a graft which produces or displays a blood group B moiety. The blood group sugar can be Type 1 or Type 2. Preferably, the recipient does not possess an enzyme which promotes the formation of a blood group B carbohydrate, e.g., UDP-GalNAc:Fucα1,2Gal-Rα1,3Gal transferase (EC 2.4.1.37), or an enzyme of equivalent activity, or does not produce or display a group B carbohydrate moiety on its cells, tissues, or organs.

The method includes:

providing to the recipient mammal a tolerance-inducing blood group B carbohydrate antigen, thereby inducing tolerance to the blood group B moiety or to a graft which includes that moiety. Although not wishing to be bound by theory, the inventors believe the carbohydrate moiety mediates the deletion of immune cells which would give rise to blood group B-reactive antibodies.

The donor can be, for example, an animal which has or expresses an allele which results in production or display of the antigen and the recipient can be an animal that lacks, or fails to express, an allele which results in production or display of the antigen.

In preferred embodiments a blood group B carbohydrate moiety is produced or displayed on a modified cell of the recipient, wherein the cell has been modified to produce or display the blood group B carbohydrate moiety. The cell can be modified in vivo or ex vivo.

In preferred embodiments: the cell is modified to produce or display a blood group B carbohydrate moiety by inserting into the cell a nucleic acid which encodes a protein which promotes, e.g., catalyzes, the formation of the blood group B carbohydrate moiety, e.g., UDP-GalNAc:Fucα1,2Gal-Rα1,3Gal transferase (EC 2.4.1.37), or an enzyme of equivalent activity.

In other preferred embodiments, the providing step of the method includes: removing the recipient mammalian cell from the recipient mammal prior to introducing nucleic acid into the recipient mammal cell and administering the recipient mammalian cell to the recipient mammal.

In preferred embodiments: the cell is modified to produce or display a blood group B carbohydrate moiety by forming the blood group B carbohydrate moiety on the surface of a cell of the recipient mammal, e.g., by contacting the cell with a protein, e.g., an enzyme which promotes the formation of the blood group B carbohydrate moiety on the surface of the cell. In particularly preferred embodiments the moiety is formed by the addition of a blood group B carbohydrate, on the surface of the recipient cell, by contacting the cell with an enzyme which promotes the synthesis or attachment of the moiety, e.g., UDP-GalNAc:Fucα1,2Gal-Rα1,3Gal transferase (EC 2.4.1.37), or an enzyme of equivalent activity.

In preferred embodiments, the method includes inactivating immune system cells, e.g., antigen-reactive immune cells, e.g, blood group B carbohydrate moiety-reactive immune cells, of the recipient, preferably prior to providing the recipient cell which produces or displays a blood group B carbohydrate moiety.

In preferred embodiments, the method includes: inactivating antibodies, e.g., antigen-reactive antibodies, e.g, blood group B carbohydrate-reactive antibodies, of the recipient, preferably prior to providing the recipient cell which produces or displays a blood group B carbohydrate moiety.

In preferred embodiments the method inhibits hyperacute rejection.

The recipient cell can be any cell suitable for presentation of a blood group B carbohydrate moiety, e.g., a hematopoietic cell. Hematopoietic stem cells, e.g., bone marrow cells, which are capable of developing into mature myeloid and/or -lymphoid cells, are particularly preferred. It is possible that later stage cells can be used. Stem cells derived from the cord blood of the recipient can be used in methods of the invention. Other cells suitable for use in the invention include peripheral blood cells. Suitable cells are those which can produce or display the blood group carbohydrate moiety and tolerize the animal. Although not wishing to be bound by theory, the inventors believe that suitable recipient cells are cells which produce or display the blood group carbohydrate moiety such that the moiety can interact with immune cells at an early stage in their development. Although not wishing to be bound by theory, this is believed to allow deletion of cells which would give rise to blood group-B reactive antibodies. Suitable cells are those which result in tolerance as opposed to an immune response.

In preferred embodiments, the method includes an additional step which inactivates a recipient anti-blood group B carbohydrate antibody. For example, anti-blood group B carbohydrate antibody activity can be inactivated prior to the introduction or formation in the recipient of a recipient cell which produces or displays group B carbohydrate moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-group B carbohydrate antibody; depleting natural antibodies from the blood of the recipient, e.g., by hemoperfusing an organ, e.g., a liver or kidney, obtained from a mammal of the donor species or by contacting the blood of the recipient with blood group B moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method further includes providing to the recipient, and inducing tolerance to, a second antigen. The antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments more than one blood group carbohydrate moiety is provided to the recipient. The second carbohydrate moiety can be provided by a method described herein.

In preferred embodiments, the method further includes: introducing a graft from a donor mammal into the recipient mammal.

In another aspect, the invention features, a method of promoting, in a recipient mammal, e.g., a human, tolerance to a blood group H carbohydrate antigen or to a graft which produces or displays a blood group H moiety. The blood group sugar can be Type 1 or Type 2. Preferably, the recipient does not possess an enzyme which promotes the formation of a blood group H carbohydrate, e.g., GDP-Fuc:βgalactosideoα2-Fuc-transferase (EC 2.4.1.69), or an enzyme of equivalent activity, or does not produce or display a group H carbohydrate moiety on its cells, tissues, or organs.

The method includes:

providing to the recipient mammal a tolerance-inducing blood group H carbohydrate antigen, thereby inducing tolerance to the blood group H moiety or to a graft which produce or displays that moiety. Although not wishing to be bound by theory, the inventors believe the carbohydrate moiety mediates the deletion of immune cells which would give rise to blood group H-reactive antibodies.

The donor can be, for example, an animal which has or expresses an allele which results in production or display of the antigen and the recipient can be an animal that lacks, or fails to express, an allele which results in production or display of the antigen.

In preferred embodiments a blood group H carbohydrate moiety is produced or displayed on a modified cell of the recipient, wherein the cell has been modified to produce or display the blood group H carbohydrate moiety. The cell can be modified in vivo or ex vivo.

In preferred embodiments: the cell is modified to produce or display a blood group H carbohydrate moiety by inserting into the cell a nucleic acid which encodes a protein which promotes, e.g., catalyzes, the formation of the blood group H carbohydrate moiety, e.g., GDP-Fuc:βgalactosideoα2-Fuc-transferase (EC 2.4.1.69), or an enzyme of equivalent activity.

In other preferred embodiments, the providing step of the method includes: removing the recipient mammalian cell from the recipient mammal prior to introducing nucleic acid into the recipient mammal cell and administering the recipient mammalian cell to the recipient mammal.

In preferred embodiments: the cell is modified to produce or display a blood group H carbohydrate moiety by forming the blood group H carbohydrate moiety on the surface of a cell of the recipient mammal, e.g., by contacting the cell with a protein, e.g., an enzyme which promotes the formation of the blood group H carbohydrate moiety on the surface of the cell. In particularly preferred embodiments the moiety is formed by the addition of a blood group H carbohydrate, on the surface of the recipient cell, by contacting the cell with an enzyme which promotes the synthesis or attachment of the moiety, e.g., GDP-Fuc:βgalactosideα2-Fuc-transferase (EC 2.4.1.69), or an enzyme of equivalent activity.

In preferred embodiments, the method includes inactivating immune system cells, e.g., antigen-reactive immune cells, e.g, blood group H carbohydrate moiety-reactive immune cells, of the recipient, preferably prior to providing the recipient cell which produces or displays a blood group H carbohydrate moiety.

In preferred embodiments, the method includes: inactivating antibodies, e.g., antigen-reactive antibodies, e.g, blood group H carbohydrate-reactive antibodies, of the recipient, preferably prior to providing the recipient cell which produces or displays a blood group H carbohydrate moiety.

In preferred embodiments the method inhibits hyperacute rejection.

The recipient cell can be any cell suitable for presentation of a blood group H carbohydrate moiety, e.g., a hematopoietic cell. Hematopoietic stem cells, e.g., bone marrow cells, which are capable of developing into mature myeloid and/or lymphoid cells, are particularly preferred. It is possible that later stage cells can be used. Stem cells derived from the cord blood of the recipient can be used in methods of the invention. Other cells suitable for use in the invention include peripheral blood cells. Suitable cells are those which can produce or display the blood group carbohydrate moiety and tolerize the animal. Although not wishing to be bound by theory, the inventors believe that suitable recipient cells are cells which produce or display the blood group carbohydrate moiety such that the moiety can interact with immune cells at an early stage in their development. Although not wishing to be bound by theory, this is believed to allow deletion of cells which would give rise to blood group H reactive antibodies. Suitable cells are those which result in tolerance as opposed to an immune response.

In preferred embodiments, the method includes an additional step which inactivates a recipient anti-blood group H carbohydrate antibody. For example, anti-blood group H carbohydrate antibody activity can be inactivated prior to the introduction or formation in the recipient of a recipient cell which produces or displays group H carbohydrate moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-group H carbohydrate antibody; depleting natural antibodies from the blood of the recipient, e.g., by hemoperfusing an organ, e.g., a liver or kidney, obtained from a mammal of the donor species or by contacting the blood of the recipient with blood group H moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method further includes providing to the recipient, and inducing tolerance to, a second antigen. The antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments more than one blood group carbohydrate moiety is provided to the recipient. The second carbohydrate moiety can be provided by a method described herein.

In preferred embodiments, the method further includes: introducing a graft from a donor mammal into the recipient mammal.

In another aspect, the invention features, a method of promoting, in a recipient mammal, e.g., a human, tolerance to a blood group Le carbohydrate antigen or to a graft which produces or displays a blood group Le moiety. Preferably, the recipient does not possess an enzyme which promotes the formation of a blood group Le carbohydrate, e.g, express GDP-Fuc:Galβ1,3/4GlcNAc-Rα4/3Fuc transferase (EC 2.4.1.65), or an enzyme of equivalent activity, or does not produce or display a group Le carbohydrate moiety on its cells, tissues, or organs.

The method includes:

providing to the recipient mammal a tolerance-inducing blood group Le carbohydrate antigen, thereby inducing tolerance to the blood group Le moiety or to a graft which produce or displays that moiety. Although not wishing to be bound by theory, the inventors believe the carbohydrate moiety mediates the deletion of immune cells which would give rise to blood group Le-reactive antibodies.

The donor can be, for example, an animal which has or expresses an allele which results in production or display of the antigen and the recipient can be an animal that lacks, or fails to express, an allele which results in production or display of the antigen.

In preferred embodiments a blood group Le carbohydrate moiety is produced or displayed on a modified cell of the recipient, wherein the cell has been modified to produce or display the blood group Le carbohydrate moiety. The cell can be modified in vivo or ex vivo.

In preferred embodiments: the cell is modified to produce or display a blood group Le carbohydrate moiety by inserting into the cell a nucleic acid which encodes a protein which promotes, e.g., catalyzes, the formation of the blood group Le carbohydrate moiety, e.g., GDP-Fuc:Galβ1,3/4GlcNAc-Rα4/3Fuc transferase (EC 2.4.1.65), or an enzyme of equivalent activity.

In other preferred embodiments, the providing step of the method includes: removing the recipient mammalian cell from the recipient mammal prior to introducing nucleic acid into the recipient mammal cell and administering the recipient mammalian cell to the recipient mammal.

In preferred embodiments: the cell is modified to produce or display a blood group Le carbohydrate moiety by forming the blood group Le carbohydrate moiety on the surface of a cell of the recipient mammal, e.g., by contacting the cell with a protein, e.g., an enzyme which promotes the formation of the blood group Le carbohydrate moiety on the surface of the cell. In particularly preferred embodiments the moiety is formed by the addition of a blood group Le carbohydrate, on the surface of the recipient cell, by contacting the cell with an enzyme which promotes the synthesis or attachment of the moiety, e.g., GDP-Fuc:Galβ1,3/4GlcNAc-Rα4/3Fuc transferase (EC 2.4.1.65), or an enzyme of equivalent activity.

In preferred embodiments, the method includes inactivating immune system cells, e.g., antigen-reactive immune cells, e.g, blood group Le carbohydrate moiety-reactive immune cells, of the recipient, preferably prior to providing the recipient cell which produces or displays a blood group Le carbohydrate moiety.

In preferred embodiments, the method includes: inactivating antibodies, e.g., antigen-reactive antibodies, e.g, blood group Le carbohydrate-reactive antibodies, of the recipient, preferably prior to providing the recipient cell which produces or displays a blood group Le carbohydrate moiety.

In preferred embodiments the method inhibits hyperacute rejection.

The recipient cell can be any cell suitable for presentation of a blood group Le carbohydrate moiety, e.g., a hematopoietic cell. Hematopoietic stem cells, e.g., bone marrow cells, which are capable of developing into mature myeloid and/or lymphoid cells, are particularly preferred. It is possible that later stage cells can be used. Stem cells derived from the cord blood of the recipient can be used in methods of the invention. Other cells suitable for use in the invention include peripheral blood cells. Suitable cells are those which can produce or display the blood group carbohydrate moiety and tolerize the animal. Although not wishing to be bound by theory, the inventors believe that suitable recipient cells are cells which produce or display the blood group carbohydrate moiety such that the moiety can interact with immune cells at an early stage in their development. Although not wishing to be bound by theory, this is believed to allow deletion of cells which would give rise to blood group Le reactive antibodies. Suitable cells are those which result in tolerance as opposed to an immune response.

In preferred embodiments, the method includes an additional step which inactivates a recipient anti-blood group Le carbohydrate antibody. For example, anti-blood group Le carbohydrate antibody activity can be inactivated prior to the introduction or formation in the recipient of a recipient cell which produces or displays group Le carbohydrate moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-group Le carbohydrate antibody; depleting natural antibodies from the blood of the recipient, e.g., by hemoperfusing an organ, e.g., a liver or kidney, obtained from a mammal of the donor species or by contacting the blood of the recipient with blood group Le moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method further includes providing to the recipient, and inducing tolerance to, a second antigen. The antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments more than one blood group carbohydrate moiety is provided to the recipient. The second carbohydrate moiety can be provided by a method described herein.

In preferred embodiments, the method further includes: introducing a graft from a donor mammal into the recipient mammal.

In another aspect, the invention features, a method of promoting, in a recipient mammal, e.g., a human, tolerance to a blood group I carbohydrate antigen or to a graft which produces or displays a blood group I moiety. Preferably, the recipient does not possess an enzyme which promotes the formation of a blood group I carbohydrate, e.g, express UDP-GlcNAc:GlcNAcβ1,3Galβ1,4GlcNAc-Rβ6-GlcNAc transferase, or an enzyme of equivalent activity, or does not produce or display a group I carbohydrate moiety on its cells, tissues, or organs.

The method includes:

providing to the recipient mammal a tolerance-inducing blood group I carbohydrate antigen, thereby inducing tolerance to the blood group I moiety or to a graft which includes that moiety. Although not wishing to be bound by theory, the inventors believe the carbohydrate moiety mediates the deletion of immune cells which would give rise to blood group 1-reactive antibodies.

The donor can be, for example, an animal which has or expresses an allele which results in production or display of the antigen and the recipient can be an animal that lacks, or fails to express, an allele which results in production or display of the antigen.

In preferred embodiments a blood group I carbohydrate moiety is produced or displayed on a modified cell of the recipient, wherein the cell has been modified to produce or display the blood group I carbohydrate moiety. The cell can be modified in vivo or ex vivo.

In preferred embodiments: the cell is modified to produce or display a blood group I carbohydrate moiety by inserting into the cell a nucleic acid which encodes a protein which promotes, e.g., catalyzes, the formation of the blood group I carbohydrate moiety, e.g., UDP-GlcNAc:GlcNAcβ1,3Galβ1,4GlcNAc-Rβ6-GlcNAc transferase, or an enzyme of equivalent activity.

In other preferred embodiments, the providing step of the method includes: removing the recipient mammalian cell from the recipient mammal prior to introducing nucleic acid into the recipient mammal cell and administering the recipient mammalian cell to the recipient mammal.

In preferred embodiments: the cell is modified to produce or display a blood group I carbohydrate moiety by forming the blood group I carbohydrate moiety on the surface of a cell of the recipient mammal, e.g., by contacting the cell with a protein, e.g., an enzyme which promotes the formation of the blood group I carbohydrate moiety on the surface of the cell. In particularly preferred embodiments the moiety is formed by the addition of a blood group I carbohydrate, on the surface of the recipient cell, by contacting the cell with an enzyme which promotes the synthesis or attachment of the moiety, e.g., UDP-GlcNAc:GlcNAcβ1,3Galβ1,4GlcNAc-Rβ6-GlcNAc transferase, or an enzyme of equivalent activity.

In preferred embodiments, the method includes inactivating immune system cells, e.g., antigen-reactive immune cells, e.g, blood group I carbohydrate moiety-reactive immune cells, of the recipient, preferably prior to providing the recipient cell which produces or displays a blood group I carbohydrate moiety.

In preferred embodiments, the method includes: inactivating antibodies, e.g., antigen-reactive antibodies, e.g, blood group I carbohydrate-reactive antibodies, of the recipient, preferably prior to providing the recipient cell which produces or displays a blood group I carbohydrate moiety.

In preferred embodiments the method inhibits hyperacute rejection.

The recipient cell can be any cell suitable for presentation of a blood group I carbohydrate moiety, e.g., a hematopoietic cell. Hematopoietic stem cells, e.g., bone marrow cells, which are capable of developing into mature myeloid and/or lymphoid cells, are particularly preferred. It is possible that later stage cells can be used. Stem cells derived from the cord blood of the recipient can be used in methods of the invention. Other cells suitable for use in the invention include peripheral blood cells. Suitable cells are those which can produce or display the blood group carbohydrate moiety and tolerize the animal. Although not wishing to be bound by theory, the inventors believe that suitable recipient cells are cells which produce or display the blood group carbohydrate moiety such that the moiety can interact with immune cells at an early stage in their development. Although not wishing to be bound by theory, this is believed to allow deletion of cells which would give rise to blood group I reactive antibodies. Suitable cells are those which result in tolerance as opposed to an immune response.

In preferred embodiments, the method includes an additional step which inactivates a recipient anti-blood group I carbohydrate antibody. For example, anti-blood group I carbohydrate antibody activity can be inactivated prior to the introduction or formation in the recipient of a recipient cell which produces or displays group I carbohydrate moieties. Thus, in preferred embodiments, the method includes one or more of: administering anti-idiotypic antibodies (e.g., recombinant, monoclonal, polyclonal, chimeric, single chain, or humanized antibodies), or fragments thereof, specific for an anti-group I carbohydrate antibody; depleting natural antibodies from the blood of the recipient, e.g., by hemoperfusing an organ, e.g., a liver or kidney, obtained from a mammal of the donor species or by contacting the blood of the recipient with blood group I moieties coupled to an insoluble substrate; administering to the recipient drugs which inactivate natural antibodies, e.g., deoxyspergualin (DSG) (Bristol); or administering to the recipient anti-IgM antibodies.

In preferred embodiments the method further includes providing to the recipient, and inducing tolerance to, a second antigen. The antigen can be produced by or displayed on a modified cell of the recipient. The modified cell can be the same cell which produces or displays the first antigen or it can be a different cell. Generally, methods described herein for providing antigen to the recipient can be used to provide the second antigen to the recipient.

In preferred embodiments more than one blood group carbohydrate moiety is provided to the recipient. The second carbohydrate moiety can be provided by a method described herein.

In preferred embodiments, the method further includes: introducing a graft from a donor mammal into the recipient mammal.

In another aspect, the invention features, a method of inactivating recipient natural antibodies which bind to an antigen which is found on the surface of a xenograft, e.g., a carbohydrate moiety, e.g., a galactosyl α(1, 3) galactose moiety, e.g., a galactosyl α(1, 3) galactose moiety on a graft, and thereby inhibiting hyperacute rejection by administering anti-idiotypic antibodies (e.g., recombinant, monoclonal, polyclonal, chimeric, single chain, or humanized antibodies), or fragments thereof, against the natural antibody.

In preferred embodiments the method further includes implanting the graft, e.g., a kidney, liver, heart, or population of hematopoietic stem cells in the recipient.

In preferred embodiments the recipient is a human and the graft is from a swine, e.g., a miniature swine.

In preferred embodiments the method inhibits hyperacute rejection.

In another aspect, the invention features, a purified preparation of an anti-idiotypic monoclonal antibody (e.g., recombinant, monoclonal, polyclonal, chimeric, single chain, or humanized antibody), or fragments thereof, directed against a natural antibody which reacts with an antigen, e.g., a carbohydrate, e.g., an galactosyl α(1, 3) galactose moiety, present on the surface of swine cells, to which antigen humans make natural antibodies.

In another aspect, the invention features, a purified preparation of an anti-idiotypic monoclonal antibody (e.g., recombinant, monoclonal, polyclonal, chimeric, single chain, or humanized antibody), or fragments thereof, directed against an antibody which reacts with a carbohydrate moiety, an galactosyl α(1, 3) galactose moiety.

Methods described herein can also include other steps to promote acceptance of or induce tolerance to the recipient cell or to the graft.

Other preferred embodiments include: the step of, preferably prior to recipient cell transplantation, creating hematopoietic space in the recipient. The reintroduction into the recipient of engineered or otherwise modified autologous cells can be optimized by the creation of hematopoietic space. Hematopoietic space can be created by the administration of antibodies or drugs which deplete the bone marrow, e.g., by administering an inhibitor of cell proliferation, e.g., DSG, or an anti-metabolite, e.g. Brequinar, or an anti-T cell antibody, e.g., one or both of an anti-CD4 or anti-CD8 antibody. Hematopoietic space can also be created by irradiating the recipient mammal with low dose, e.g., between about 100 and 400 Rads, whole body irradiation to deplete or partially deplete the bone marrow of the recipient. The creation of hematopoietic space does not totally ablate the recipients bone marrow but allows for the production of mixed chimerism. The need for hematopoietic space can be minimized by the creation in the recipient of thymic space.

Other preferred embodiments include: the step of creating thymic space in the recipient, e.g., by irradiating the thymus of the recipient, e.g., by administering between 100 and 1,000, more preferably between 300 and 700, e.g., 700 Rads, of thymic irradiation, or by administering anti-T cell antibodies in sufficient dose to inactivate thymocytes. Other methods for the creation of thymic space include: the administration of steroids, corticosteroids, Brequinar, or immune suppressant drugs, e.g., rapamycin, cyclosporin, or FK506. Methods of creating thymic space are disclosed in provisional U.S. patent application No. 60/017,099, hereby incorporated by reference. The methods disclosed herein can be combined with the methods disclosed in U.S. patent application No. 60/017,099.

In preferred embodiments, the method includes: inactivating immune system cells, e.g., xenoreactive immune cells, of the recipient. Immune system cells include thymocytes, T cells, B cells, and NK cells.

In other preferred embodiments, the method includes: inactivating T cells, e.g., xenoreactive T cells, of the recipient mammal, e.g., by prior to introducing recipient cells or a graft into the recipient mammal, introducing into the recipient mammal an antibody capable of binding to T cells of the recipient mammal.

In preferred embodiments, the method includes: inactivating natural killer cells, e.g., xenoreactive NK cells, of the recipient mammal, e.g., by prior to introducing the cells or a graft into the recipient mammal, introducing into the recipient mammal an antibody capable of binding to natural killer cells of the recipient mammal.

One source of anti-NK antibody is anti-human thymocyte polyclonal anti-serum. A second anti-mature T cell antibody can be administered as well, which inactivates T cells as well as NK cells. Depletion, Inactivation of T cells is advantageous for both bone marrow and xenograft survival. Anti-T cell antibodies are present, along with anti-NK antibodies, in anti-thymocyte anti-serum. Repeated doses of anti-NK or anti-T cell antibody may be preferable. Monoclonal preparations can be used in the methods of the invention.

The methods described herein can be combined with methods of inducing tolerance described in U.S. Ser. No. 08/266,427, filed Jun. 27, 1994, the contents of which are hereby expressly incorporated by reference. Thus, the methods disclosed herein can include administering to the recipient a recipient cell which expresses a donor MHC class I gene or a donor MHC class II gene (or both). The cell which expresses the donor MHC gene can be the same cell which expresses the galactosyl α(1, 3) galactose moiety or it can be a different cell.

In preferred embodiments, a short course of help reducing treatment can be used to induce tolerance to the recipient cell or the graft. In particular, the methods described in U.S. Ser. No. 08/458,720, filed Jun. 1, 1995, the contents of which are expressly incorporated herein by reference, can be combined with the methods described herein.

In preferred embodiments, a short course of an immunosuppressive agent can be administered to inhibit T cell activity in the recipient. In particular, the methods described in U.S. Ser. No. 08/458,720, filed Jun. 1, 1995, the contents of which are expressly incorporated herein by reference, can be combined with the methods described herein.

Methods of inducing tolerance by the methods described herein can also be combined with yet other methods for inducing tolerance, e.g., with: methods which use the implantation of donor stem cells to induce tolerance, e.g., the methods described in U.S. Ser. No. 08/451,210, filed on May 26, 1995, the contents of which are hereby expressly incorporated by reference; methods which use stem cells or other tissue from genetically engineered swine, e.g., the genetically engineered swine in U.S. Ser. No. 08/292,565, filed Aug. 19, 1994, the contents of which are expressly incorporated herein by reference, or in U.S. Ser. No.08/692, 843, filed Aug. 2, 1996, the contents of which are expressly incorporated herein by reference; methods which use the implantation of a xenogeneic thymic graft to induce tolerance, e.g., the methods described in U.S. Ser. No. 08/163,912, filed on Dec. 7, 1993, the contents of which are hereby expressly incorporated by reference; methods of increasing the level of the activity of a tolerance promoting or GVHD inhibiting cytokine or decreasing the level of activity of a tolerance inhibiting or GVHD promoting cytokine, e.g., the methods described in U.S. Ser. No. 08/114,072, filed Aug. 30, 1993, the contents of which are hereby expressly incorporated by reference; methods of using cord blood cells to induce tolerance, e.g., the methods described in U.S. Ser. No. 08/150,739 filed Nov. 10, 1993, the contents of which are hereby expressly incorporated by reference; methods of preventing GVHD, e.g., the methods described in U.S. Ser. No. 08/461,693, filed Jun. 5, 1995, the contents of which are hereby expressly incorporated by reference; with methods of promoting tolerance by enhancing or maintaining thymus function, e.g., the methods described in U.S. Ser. No. 08/297,291, filed Aug. 26, 1994, the contents of which are hereby expressly incorporated by reference; methods of detecting the presence of swine retroviral sequences, e.g., the methods described in U.S. Ser. No. 08/572,645, filed Dec. 14, 1995, or a continuation of U.S. U.S. Ser. No. 08/572,645, filed Dec. 13, 1996, the contents of which are hereby expressly incorporated by reference; and the methods for inducing tolerance disclosed in Sykes and Sachs, PCT/US94/01616, filed Feb. 14, 1994, the contents of which are hereby expressly incorporated by reference.

In another aspect, the invention features a method of treating a subject mammal, e.g., a human, having a disorder characterized by an unwanted antibody directed against an autoantigen. The method includes:

providing to the mammal a tolerance-inducing autoantigen, e.g., a carbohydrate moiety, protein, or peptide, thereby inducing tolerance to the autoantigen. Although not wishing to be bound by theory, the inventors believe the autoantigen mediates the deletion of immune cells which would give rise to autoantigen-reactive antibodies.

In preferred embodiments the subject is a human and the autoantigen is one which mediates diabetes, MS, lupus, or arthritisis.

In preferred embodiments, the autoantigen is produced by or displayed on a modified cell of the subject, wherein the cell has been modified to produce or display the autoantigen. The cell can be modified in vivo (in the recipient's body), e.g., by in vivo gene therapy or by in vivo treatment with an agent which modifies the cell, or ex vivo (removed from the subject's body). The cell can be modified by inserting into the cell a nucleic acid which encodes the autoantigen, (or otherwise promotes the production or display of the autoantigen) such that the cell produces or displays the autoantigen. The cell can be modified to produce or display a carbohydrate moiety by inserting into the cell a nucleic acid encoding a protein which promotes, e.g., catalyzes, the formation of the carbohydrate moiety. The encoded protein can be an enzyme which results in the formation of a carbohydrate moiety on the surface of the cell. In particularly preferred embodiments the encoded protein forms the moiety by the addition of a terminal sugar residue to a pre-existing sugar residue on a cell surface molecule.

The cell can be modified to produce or display the autoantigen, e.g., a protein or carbohydrate moiety, by forming the autoantigen, e.g., a protein or carbohydrate moiety, in or on the surface of a cell of the recipient mammal or attaching the autoantigen to the subject cell, e.g., by contacting the cell with a protein, e.g., an enzyme, which results in the formation of the autoantigen, e.g., a carbohydrate moiety, on the surface of the cell or by adhering or attaching the autoantigen to the cell. In particularly preferred embodiments the protein forms the moiety by the addition of a terminal sugar residue to a pre-existing sugar residue on a cell surface molecule.

In preferred embodiments the cell is removed from the subject, modified so as to allow it to produce or display the autoantigen and implanted in the recipient.

In preferred embodiments, the method includes: preferably prior to providing the tolerance-inducing autoantigen, inactivating immune system cells, e.g., autoantigen-reactive immune cells, of the recipient.

In preferred embodiments, the method includes: preferably prior to providing the tolerance-inducing autoantigen inactivating antibodies, e.g., autoantigen reactive antibodies, e.g, carbohydrate moiety-reactive antibodies, of the recipient.

In preferred embodiments the method further includes providing to the recipient, and inducing tolerance to, a second autoantigen, e.g., a carbohydrate moiety, protein, or peptide. The second autoantigen can be produced by or displayed on a modified cell of the recipient. The modified cell can be the same cell which produces or displays the first autoantigen or it can be a different cell. Generally, methods described herein for providing autoantigen to the recipient can be used to provide the second autoantigen to the recipient.

“Antigen” as used herein is a molecule which can be recognized as non-self by a recipient immune system and includes proteins and carbohydrates, e.g., carbohydrates found on glycoproteins or glycolipids. Preferred antigens are those which react with natural antibodies in humans.

“Forming a galactosyl α(1,3) galactose moiety on the surface of a cell” refers to a process which results in the cell presenting a galactosyl α(1,3) galactose moiety on its surface. Forming can include attaching, preferably by a covalent modification, a galactosyl α(1,3) galactose moiety, or enzymatically forming a galactosyl α(1,3) galactose moiety, on the surface of the cell.

“Galactosyl α(1, 3) galactose epitope”, as used herein, refers to epitopes located wholly or partially on galactosyl α(1, 3) galactose structures, e.g., those located wholly or partially on galactosyl (α1,3) galactose structures of αGal (1-3)βGal (1-4)βGlcNAc or αGal (1-3)βGal (1-4)βGlc structures.

“Galactosyl α(1, 3) galactose moiety”, as used herein, refers to the galactosyl α(1,3) galactose structure, e.g., as found in αGal (1-3)βGal (1-4)βGlcNAc or αGal (1-3)βGal (1-4)βGlc structures.

“α(1,3)galactosyltransferase, e.g., β-D-galactosyl-1,4-N-acetyl-D-glucosaminide α(1,3)galactosyltransferase activity, as used herein refers to the enzymatic activity of forming galactosyl α(1, 3) galactose moieties. Enzyme activity which forms the B blood group antigen is not covered by this definition.

“Graft”, as used herein, refers to a body part, organ, tissue, or cells. Grafts may consist of organs such as liver, kidney, heart or lung; body parts such as bone or skeletal matrix; tissue such as skin, intestines, endocrine glands, thymic tissue; or progenitor stem cells of various types.

“Inactivation of an antibody (or an antibody response)” refers to a treatment which reduces the number of antibodies, particularly xenoreactive antibodies, e.g., galactosyl α(1, 3) galactose moiety reactive antibodies, which can bind their cognate epitope in a subject. Inactivation includes: removal of antibodies from the subject, e.g., by contacting the blood of the subject with a reagent, e.g., an affinity matrix, which allows removal of antibodies from the blood; inactivating an immune cell which promotes the formation of the antibody; and inhibiting an antibody by contacting it with an anti-idiotypic antibody.

“Inactivation of an immune cell” refers to a treatment which reduces the number of active immune cells, e.g., thymocytes, T cells, B cells, or NK cells in a subject. Inactivation includes: removal from the blood of the subject; temporarily or permanently inhibiting an immune cell e.g., a T or B cell by, e.g., administering a drug such as an inhibitor of cell proliferation, e.g., DSG, or an anti-metabolite, e.g. Brequinar; temporarily or permanently inhibiting an immune cell by administering an anti-immune cell antibody, e.g., an anti-T cell antibody, e.g., one or both of an anti-CD4 or anti-CD8 antibody, an anti-B cell antibody, or an anti-NK cell antibody.

“Lymph node or thymic T cell”, as used herein, refers to T cells which are resistant to inactivation by traditional methods of T cell inactivation, e.g., inactivation by a single intravenous administration of anti-T cell antibodies, e.g., antibodies, e.g., ATG preparation.

“MHC antigen”, as used herein, refers to a protein product of one or more MHC genes; the term includes fragments or analogs of products of MHC genes which can evoke an immune response in a recipient organism. Examples of MHC antigens include the products (and fragments or analogs thereof) of the human MHC genes, i.e., the HLA genes. MHC antigens in swine, e.g., miniature swine, include the products (and fragments and analogs thereof) of the SLA genes, e.g., the DRB gene.

“Miniature swine”, as used herein, refers to a miniature pig which is preferably wholly or partially inbred at at least one MHC locus. The coefficient of inbreeding of the herd which supplies the miniature swine should be at least, 0.70 and more preferably at least 0.82.

“Produces or displays”, as used herein, means that the entity, e.g., a cell, a tissue, or an organ, provides on its surface, secretes, or otherwise provides, the moiety. The moiety is accessible to one or more components of the immune system, e.g., antibodies, or cell-bound receptors, e.g., T cell receptors.

“Recipient cell” as used herein refers to a cell suitable for the tolerizing expression of the galactosyl α(1, 3) galactose epitope. For example, a recipient cell can be a hematopoietic cell, e.g., a bone marrow cell which is capable of developing into a mature myeloid and/or lymphoid cell. Stem cells derived from cord blood, bone marrow, or peripheral blood of the recipient can be used in methods of the invention. See U.S. Pat. No. 5,192,553, hereby incorporated by reference, and U.S. Pat. No. 5,004,681, hereby incorporated by reference.

“Thymic space”, as used herein, is a state created by a treatment that facilitates the migration to and/or development in the thymus of donor or engineered autologous hematopoietic cells of a type which can delete or inactivate recipient thymocytes that recognize donor antigens. It is believed that the effect is mediated by elimination of preexisting recipient cells in the thymus.

“Hyperacute rejection”, as used herein refers a recipient anti-donor response which is mediated at least in part by preformed antibodies.

“Moiety” as used herein, refers to all or part of a chemical entity, e.g., a all or part of a carbohydrate.

“Tolerance”, as used herein, refers to the inhibition of a graft recipient's immune response, particularly the hyperacute rejection response, which would otherwise occur, e.g., in response to the introduction of an antigen, e.g., galactosyl α(1, 3) galactose moiety, into the recipient. The term “tolerance” refers not only to complete immunologic tolerance to an antigen, but to partial immunologic tolerance, i.e., a degree of tolerance to an antigen which is greater than what would be seen if a method of the invention were not employed. Tolerance can involve humoral, cellular, or both humoral and cellular responses. Tolerance is specific for the antigen, e.g., galactosyl α(1, 3) galactose moiety, or epitopes which are located wholly or in part on that moiety, and does not refer to a general state of immunosuppression. Although not wishing to be bound by theory, the inventors believe tolerance may be achieved by deletion of immune which would otherwise give rise to antigen-reactive, e.g., αgalactosyl α(1,3)galactose-reactive antibodies.

Removal of xenogeneic natural antibodies using organ perfusion, or more recently using synthetic galactosyl α(1, 3) galactose columns, has been shown to delay the onset of hyperacute rejection. However, due to the continued presence of natural antibody-producing B cells, the level of natural antibodies increases after the first week of transplantation and can contribute to delayed graft rejection. Methods of the invention can be used to manipulate the natural antibody response, e.g., with gene therapy, to induce tolerance at the B cell level, thereby promoting acceptance of graft tissue.

Miniature swine are an attractive potential donor for clinical xenotransplantation because of their physiological similarity to humans and their breeding characteristics (Sachs, D. et al. (1994) Path. Biol. 42:217). However, a major obstacle to clinical xenotransplantation in discordant species combinations such as swine to primate is hyperacute graft rejection mediated by preformed natural antibodies present in the recipient (Galili, U. (1993) Immunol. Today 14:480; Platt, J. L. and Bach, F. H. (1991) Transplantation 52:937; Platt, J. L. et al. (1990) Immunol. Today 11:450).

The galactosyl (α1,3) galactose epitope is the major target of human natural antibodies (reviewed in Galili, U. (1993) Immunol. Today 14:480; Platt, J. L. and Bach, F. H. (1991) Transplantation 52:937; Platt, J. L. et al. (1990) Immunol. Today 11:450; Sandrin, M. S. and McKenzie, I. F. (1994) Immunol. Rev. 141:169). This carbohydrate epitope is synthesized by the addition of a terminal galactosyl residue to a preexisting galactose residue linked to N-acetyl-glucosaminyl residue. The reaction is catalyzed by the glucosyltransferase UDP galactose: β-D-galactosyl-1,4-N-acetyl-D-glucosaminide α(1,3)galactosyltransferase (α1,3GT). In species expressing α1,3GT, natural antibodies reactive against the galactosyl (α1,3) galactose moiety are absent. The lack of α1,3GT in humans, apes, and Old World primates results in a failure to express the galactosyl α(1,3) galactose epitope, making the presence of natural antibodies reactive to this epitope permissible. It has been shown in mice, a species that normally expresses galactosyl α(1,3) galactose epitope, that disruption of murine α1,3GT gene by embryonic stem cell technology leads to the development of natural antibodies reactive against the galactosyl α(1,3) galactose epitope (Thall, A. et al. (1995) J. Biol. Chem. 270:21437; Thall, A. et al. (1996) Transplant. Proc. 28:561). Prevention of the interaction of natural antibodies with the galactosyl α(1,3) galactose epitope has been a major goal in the field of xenotransplantation.

Several different approaches aimed at eliminating the problem of natural antibody-mediated rejection in xenotransplantation have been attempted. Manipulation of the galactosyl α(1,3) galactose epitope on donor organs by various methods as well as altering the expression of α1,3GT has been attempted (Sandrin, M. S. et al. (1995) Nat. Med. 1:1261; Rosengard, A. M. et al. (1995) Transplant. Proc. 27:326; Langford, G. A. et al. (1994) Transplant. Proc. 26:1400; LaVecchio, J. A. et al. (1995) Transplantation 60:841). A short coming of these approaches has been the failure to completely abolish the expression of the epitope. Removal of serum natural antibodies from the host by adsorption has been successful in preventing hyperacute rejection but does not result in the permanent removal of galactosyl α(1, 3) galactose reactive natural antibodies, and therefore is not a long-term solution. Modification of the host humoral system by inducing tolerance to the galactosyl α(1, 3) galactose epitope provides a viable long-term solution to the problem of galactosyl α(1, 3) galactose reactive natural antibodies.

Other features and advantages of the invention will be apparent from the following detailed description, and from the claims.

### DETAILED DESCRIPTION

### BRIEF DESCRIPTION OF THE DRAWINGS

FIG. 1 (Panels A, B, C, D) is a set of graphs which show α1,3Gal/BSA-inhibition of human natural antibody binding to pig cells. Human serum (20 μl) was preincubated with αGal/BSA (Panels A,C) or bovine thyroglobulin (Panels B,D) at concentrations of 1,000 μg/ml (b) or 0.1 μg/ml (c) prior to staining of porcine peripheral blood mononuclear cells (pPBMCs). The negative control (a) consisted of pPBMC plus an equivalent volume of galactosyl α(1, 3) galactose reactive natural antibody-depleted human serum (XNA⁻) while the positive control was 20 μl human serum without competitor (d). Following incubation with the human serum, the cells were stained with either anti-human IgG (Panels A,B) or anti-human IgM (Panels C,D).

FIG. 2 (Panels A, B) is a set of graphs which show galactosyl α(1, 3) galactose reactivity of human serum. Serial dilutions (1:2, 1:4, 1:8, 1:16, 1:32, 1:64, 1:128) of serum samples from twelve unrelated donors were analyzed for binding of IgG (A) or (IgM) (B) to αGal/BSA. The serum samples are designated by donor number.

FIG. 3 (Panels A, B) is a set of graphs which show low expression of galactosyl α(1, 3) galactose reactive natural antibodies in individuals with the blood group B antigen Serially diluted human serum from B and non-B expressing donors were analyzed by αGal/BSA ELISA. IgM (Panel A) and IgG (Panel B) binding is expressed an mean optical density (O.D.) versus serum dilution. Solid bars=A,O serum; filled bars=B,AB serum.

FIG. 4 (Panels A, B) is a set of graphs which show that human natural antibodies from unrelated donors express a crossreactive idiotype. binding of IgG (Panel A) and IgM (Panel B) to αGal/BSA was evaluated by ELISA in the absence (filled bars) and in the presence (open bars) of 10784 anti-idiotype reagent. Percent inhibition was calculated for each donor.

FIG. 5 is a diagram of the LGTA7 and MZGT retroviral vectors.

FIG. 6 is a graph of the analysis of anti α(1-3)Gal reactive IgM antibodies in mice reconstituted with LGTA7 or Neo transduced bone marrow at 12 weeks post bone marrow transplantation by ELISA. All assays were performed using α(1-3)Gal conjugated to bovine serum albumin to coat ELISA plate wells. In all assays, background binding observed using serum from normal unreconstituted mice was subtracted. Similar results were obtained by subtracting the background binding observed on lactosamine coated plates. The background binding observed with serum from the LGTA7 transduced group is not α(1-3)Gal specific as shown in FIG. 7. Similar results were observed at 18 weeks.

FIG. 7 is a graph of an analysis of serum antibodies capable of lysing α(1-3)Gal positive porcine PK-15 in the presence of rabbit complement. P value between groups of mice reconstituted with Neo of LGTA7 transduced bone marrow is shown. Sera was analyzed 9 weeks post bone marrow transplantation.

### THE GALACTOSYL(α1,3)GALACTOSE MOIETY

Galactosyl α(1, 3) galactose reactive natural antibodies are important in the process of hyperacute rejection. The determinants recognized by human anti-pig natural antibodies appear to be expressed in all tissues, including lymphocytes. The terminal galactosyl α(1, 3) galactose carbohydrate structure, responsible for most of the human anti-pig response, is synthesized by all mammals with the exception of humans and Old World primates. More than 80% of the xenoreactive natural antibodies in human serum are specifically reactive with galactosyl α(1, 3) galactose, suggesting that the majority of this population of human natural antibodies is highly restricted in terms of antigen specificity. The removal of galactosyl α(1, 3) galactose reactive natural antibodies from the sera of recipient monkeys by column perfusion eliminates hyperacute rejection.

The gene encoding the enzyme responsible for the galactosyl α(1, 3) galactose structure, α((1,3)galactosyltransferase (α1,3GT) is non-functional in humans and Old World monkeys. The inactivation of α1,3GT is estimated to have occurred some 28 million years ago. Neither human nor Old World monkey derived cells are reactive with natural antibodies. Conversely, the expression of murine or porcine α1,3GT in COS cells (Old World Monkey) results in the production of the galactosyl α(1, 3) galactose epitope which is recognized by anti-galactosyl α(1,3) galactose natural antibodies. Thus, susceptibility to hyperacute rejection is determined by expression of a functional α1,3GT rather than simply by phylogenetic distance between animals. This was demonstrated by transplantation studies between New World and Old World monkeys. When the heart of a New World monkey was transplanted into an Old World monkey, the graft ceased functioning within one hour. Consistent with natural antibody mediated hyperacute rejection, immunopathologic studies revealed the presence of immunoglobulin deposits in the graft.

The selective advantage conferred by the loss of α((1,3)galactosyltransferase and subsequent anti-galactosyl α(1, 3) galactose antibody production in human and Old World primates is not known. The loss of the enzyme activity and the suppression of this epitope may be related to the production of anti-galactosyl α(1, 3) galactose antibody. There may be a role for these antibodies in protection against the transmission of C-type retroviruses from discordant species. According to published reports, galactosyl α(1, 3) galactose reactive natural antibodies comprise approximately 1% of the circulating Ig in healthy individuals and thus represent a significant barrier to xenotransplantation.

Formation of Galactosyl(α1,3)Galactose Epitopes on Recipient Cells

Genetically Engineering Cells to Present Galactosyl α(1, 3) Galactose Moieties

Nucleic acid encoding a protein which promotes the formation of the galactosyl α(1, 3) galactose epitope can be introduced into the recipient cells by any method which allows expression of the nucleic acid at a level and for a period sufficient to confer tolerance. These methods include, by way of example, transfection, electroporation, particle gun bombardment, and transduction by viral vectors, e.g., by retroviruses.

Some classical methods for introducing genes in mammalian cells have a limited efficiency which limits their usefulness in many systems (Hwang, L. H. et al. (1984) J. Virol. 50:417). Recombinant retroviruses have therefore been developed as vehicles for gene transfer (Eglitis, M. A. et al. (1988) Adv. Exp. Med. Biol. 241:19; Anderson, W. F. (1992) Hum. Gene Ther. 3:1; al-Lebban, Z. S. et al. (1990) Exp. Hematol. 18:180). The most straightforward retroviral vector construct is one in which the structural genes of the virus are replaced by a single gene which is then transcribed under the control of regulatory elements contained in the viral long terminal repeat (LTR) (Blair, D. G. et al. (1980) Proc. Natl. Acad. Sci. USA 77:3504). A variety of single-gene-vector backbones have been used, including the Moloney murine leukemia virus (MoMuLV). Derived from this type of backbone are retroviral vectors into which multiple genes, e.g., a selectable marker and a gene of interest both under the control of an internal promoter, can be inserted (McLachlin, J. R. et al. (1990) Prog. Nucleic Acid Res. Mol. Biol 38:91).

Use of efficient packaging cell lines has increased both the efficiency and spectrum of infectivity of the recombinant virions produced (Miller, A. D. (1989) Biotechniques 7:980). Following transduction with these retroviruses, the most efficient expression was observed when “strong” promoters were used to control transcription of the introduced genes separately from the viral transcription initiated within the LTR (Chang, J. M. et al. (1989) Int. J. Cell Cloning 7:264). The major limitation of this strategy has been that the second transcriptional unit containing the transduced gene was placed within the retroviral transcriptional unit, causing transcriptional interferences (Emerman, M. et al. (1984) Cell 39:449; Kadesch, T. et al. (1986) Mol. Cell Biol. 6:2593; Cullen, B. R. et al. (1984) Nature 307:241). Results from different laboratories suggest that the outcome of placing promoter elements internal to the LTR is somewhat unpredictable; in some cases leading to efficient transcription (Garver, R. I. et al. (1987) Proc. Natl. Acad. Sci. USA 84:1050) and in other cases resulting in weak or absent expression (Dzierzak, E. A. et al. (1988) Adv. Exp. Med. Biol. 241:41; Williams, D. A. et al. (1986) Proc. Natl. Acad. Sci USA 83:2566). A new type of retroviral vector, called the double-copy (DC) vector, has been developed to overcome this problem by physically separating the viral and non-viral transcription units (Hantzopoulos, P. A. et al. (1989) Proc. Natl. Acad. Sci. USA 86:3519). In addition, the DC vector allows multiple insertions and leads to efficient expression in human lymphocytes. Such retroviruses represent the best technology available at present for the transfer of genes that may prove to be clinically relevant.

In situ formation of galactosyl α(1, 3) galactose epitopes

Galactosyl α(1,3) galactose moieties can be added, in situ, to the surface of recipient cells. For example, recipient cells can be removed from the recipient and incubated with an enzyme which promotes the formation of galactosyl α(1,3) galactose moieties on the cell. See, e.g., LaTemple et al., 1996, Cancer Res. 56:3069-3074, which is hereby incorporated by references, which discloses the use of recombinant alpha 1,3 galactosyltransferase to synthesize galactosyl α(1,3) galactoside epitopes on human cells, in vitro; Josiasse et al., 1990, Eur. J. Biochem. 191:75-83, which is hereby incorporated by reference, which describes the production of recombinant enzyme; and Hamadeh et al., 1996, Infect. Immun. 64:528-534, which is hereby incorporated by reference, which describes the use of bacterial enzymes to form alpha 1,3gal structures on human cells.

The invention is further illustrated by the following examples which in no way should be construed as being further limiting. The contents of all cited references (including literature references, issued patents, published patent applications, and co-pending patent applications) cited throughout this application are hereby expressly incorporated by reference.

### EXAMPLES

### Example 1

### The Majority of Natural Antibodies Present in Human Serum are Reactive with the Galactosyl α(1,3) Galactose Moiety

A competitive binding assay was used to demonstrate that the natural antibodies recognize the galactosyl α(1, 3) galactose moiety.

Human sera were obtained from healthy adult volunteers. Individual samples were isolated and stored at 4° C. for short periods or aliquoted and stored at −70° C. Porcine peripheral blood mononuclear cells (pPBMC) were isolated from heparinized whole blood obtained from an inbred miniature swine herd and resuspended in flow cytometric analysis staining buffer (Hank's Buffered Saline Solution (HBSS), 2.0% fetal calf serum, 0.04% sodium azide (Sigma Chemical Co., St. Louis, Mo.)) to a final concentration of 1×10⁷ pPBMC/ml.

Human serum (15 μl) was pre-incubated with serially diluted αGal/BSA, bovine thyroglobulin (Sigma Chemical Co., St. Louis, Mo.) or BSA (Fisher Scientific, Pittsburgh, Pa.) at final concentrations of 1 mg/ml to 1 ng/ml for 90 minutes at 4° C. with gentle rocking. Fifty μl of pPBMC (5×10⁵ cells) was incubated with 50 μl of human serum plus competitor for 90 minutes at 4° C. The positive control consisted of staining with human serum alone and the negative control was human serum depleted of natural antibodies (XNA⁻). The cells were processed for all subsequent steps in the same way as for direct flow cytometric analysis (DerSimonian, H. M. et al. (1993) J. Exp. Med. 177:1623).

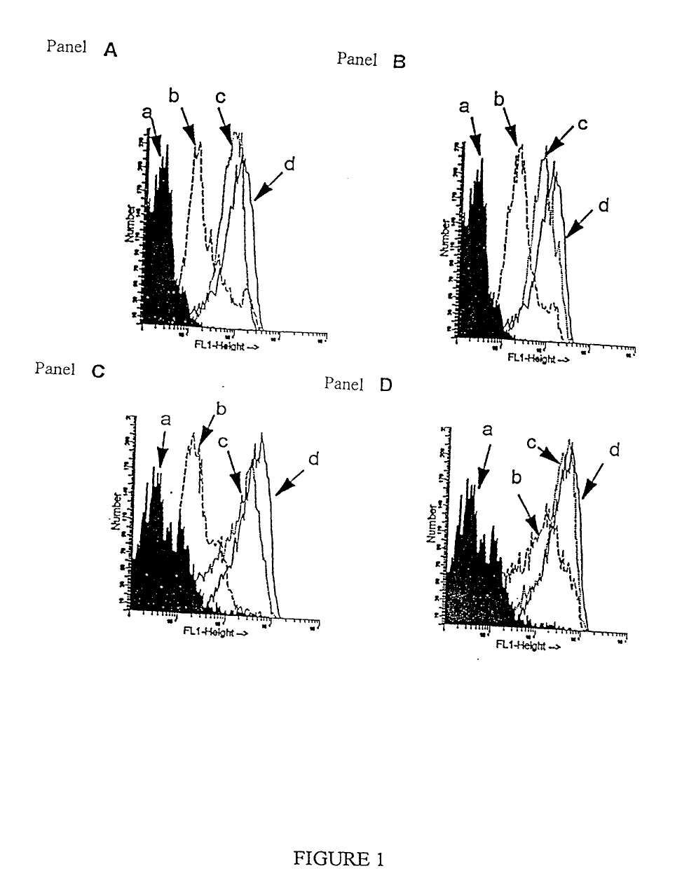

When human serum was pre-incubated with αGal/BSA, the binding of natural antibodies to porcine PBMC, as determined by median fluorescence intensity (M.F.I.), was substantially reduced. At an αGal/BSA concentration of 1 mg/ml, a 94% reduction of IgM binding to pig cells was observed; with IgG there was a 84% decrease in binding (FIG. 1). With lower concentrations of αGal/BSA, the level of immunoglobulin binding increased. Pre-incubation of human antibodies with another galactosyl α(1, 3) galactose containing molecule, bovine thyroglobulin, also had an inhibitory effect on the binding of human IgG and human IgM to porcine PBMC. Bovine thyroglobulin at a concentration of 1 mg/ml reduced the binding of antibodies to porcine cells by 66% for IgM and 79% for IgG (FIG. 1). The bovine thyroglobulin molecule contains an estimated eleven naturally occurring galactosyl α(1, 3) galactose residues (Thall, A. and Galili, U. (1990) Biochemistry 29:3959), while the synthetic conjugate, αGal/BSA, has from fifteen to thirty-nine αGal moieties per BSA molecule. In addition, αGal/BSA has a smaller molecular size (70 kD) than bovine thyroglobulin (300 kD). Consequently, αGal/BSA competes more effectively with natural antibodies for binding to pig cells than bovine thyroglobulin. No inhibition was seen when human serum was pre-incubated with BSA under the same conditions. These observations have implicated galactosyl α(1, 3) galactose as the determinant recognized by the majority of xeno-reactive natural antibodies, consistent with the observations of other groups (Collins, B. H. et al. (1995) J. Immunol. 154:5500; Oriol, R. et al. (1993) Transplantation 56:1433; Sandrin, M. S. et al. (1993) Proc. Natl. Acad. Sci. USA 90:11391; Cooper, D. K. et al. (1993) Transplant. Immunol. 1: 198; Parker, W. et al. (1994) J. Immunol. 153:3791).

### Example 2

### Determination of the Amounts of IgG and IgM in Human Sera

In order to facilitate the detection of galactosyl α(1, 3) galactose-reactive antibodies in human serum samples, an ELISA system using αGal/BSA as the antigen was used. αGal/BSA (provided by BioTransplant, Inc.) was used to coat 96 well polystyrene plates (Costar) at a concentration of 10 μg/ml in PBS overnight at 4° C. Plates coated with BSA were included to control for background reactivity to BSA. Wells were blocked for 1-2 hours at room temperature with 1% BSA in TBS (100 mM Tris-HCl, pH 7.5, 0.9% NaCl). All plates were washed three times with 0.1% Tween-20 in TBS (TBS/Tween) using a Skatron plate washer (Skatron Instruments, Inc., Sterling, Va.). Serial dilutions of human serum from a single donor were used as the standard positive controls. XNA⁻ (see Example 3) was used as the negative control. Samples were aliquoted in triplicate and allowed to incubate at room temperature for 1 hour. After washing three times with TBS/Tween, the plates were incubated with alkaline phosphatase conjugated mouse monoclonal anti-human IgG or IgM antibody (Sigma Chemical Co., St. Louis, Mo.) for 1-2 hours at room temperature in the dark. The plates were washed and developed with Sigma 104 alkaline phosphatase substrate tablets in carbonate buffer (0.203 g MgCl₂, 2.2 g Na₂CO₃, 2.43 g. NaHCO₃ in 1 L. water). The amount of colored product was measured at 405 nm using the SLT Lab Instruments 340 ATC ELISA reader. For competitive ELISA, bovine thyroglobulin and αGal/BSA were serially diluted ten-fold in TBS to final concentrations of 1 mg/ml to 1 ng/ml. Equal volumes of human serum and competitor were combined and incubated for 1 hour at 4° C. The competition reactions were then aliquoted to antigen coated plates and incubated for 1 hour at room temperature. The plates were washed and incubated with alkaline phosphatase conjugated mouse monoclonal anti-human IgG or IgM antibody (Sigma Chemical Co., St. Louis, Mo.) for 1 hour at room temperature. The wells were washed and developed with Sigma 104 alkaline phosphatase substrate. All samples were analyzed in triplicate.

To ensure that the galactosyl α(1, 3) galactose epitope was stable under these conditions, experiments were performed to determine if there was any detectable hydrolysis of the galactosyl α(1, 3) galactose carbohydrate moiety in the presence of human serum. Although all the assays were carried out at 24° C., it was found that αGal/BSA was stable in this ELI SA system even when the assay was carried out at 37° C. for up to 24 hours.

The specificity of this ELISA was demonstrated through competition with bovine thyroglobulin. The binding of serum natural antibodies to αGal/BSA were completely inhibited by pre-incubation with bovine thyroglobulin. Again, αGal/BSA was a more effective competitor than bovine thyroglobulin since it completely inhibited the binding of human IgG and IgM to bovine thyroglobulin at 1 μg/ml, while a higher concentration (100-1,000 μg/ml) of bovine thyroglobulin was necessary to block binding of αGal-reactive natural antibodies to αGal/BSA.

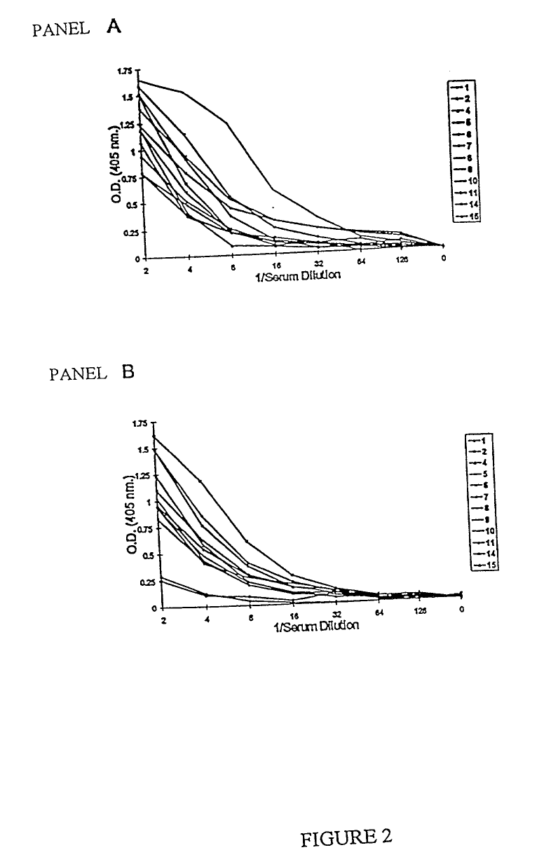

Having determined that this ELISA system could be used to assess the galactosyl α(1, 3) galactose-reactive natural antibody levels in human serum, samples from more than 95 unrelated donors were assayed in this manner. IgG and IgM concentrations were determined by competitive ELISA (Ali, R. et al. (1985) Mol. Immunol. 22:1415) using commercial IgG and IgM (Sigma Chemical Co., St. Louis, Mo.) as standards. In the ELISA results for 12 representative donors, and all others which have been tested, the relative IgG and IgM αGal/BSA reactivity varied substantially between donors; for these 12 samples there was approximately an eight-fold range in galactosyl α(1, 3) galactose reactivity. (FIG. 2)

### Example 3

### Characterization of Affinity Purified Natural Antibodies

Human galactosyl α(1, 3) galactose-reactive XNAs were affinity purified from human serum using an galactosyl α(1, 3) galactose column. Beads coupled with galactosyl α(1, 3) galactose were used to affinity purify galactosyl α(1, 3) galactose-reactive natural antibodies. Columns containing these beads (10 ml) were washed extensively with PBS and then loaded with 10 ml human plasma. Samples were loaded and allowed to run at a flow rate of 30 ml per hour, followed by washing with 8 column volumes of PBS. Bound antibodies were eluted with 100 mM sodium citrate, pH 3 and immediately neutralized with 1M Tris-HCl, pH 8.0. One ml fractions were collected and assayed for total IgG and IgM concentrations as well as for galactosyl α(1, 3) galactose specificity. Prior to assaying either the αGal/BSA or pig cell reactivity of the column fractions, any dilution of the flowthrough or concentration of the eluate was compensated for relative to the original plasma. That galactosyl α(1, 3) galactose column removed all of the detectable galactosyl α(1, 3) galactose-reactive IgG and IgM was seen by analysis of the flowthrough and wash fractions. In contrast, the renatured eluate fractions had substantial levels of αGal/BSA reactive IgG and IgM. The flowthrough and wash fractions contained high levels of IgG and IgM which gradually decreased throughout the wash fractions and increased only slightly in the eluate fractions indicating that all of the αGal/BSA reactivity was present in the small fraction of IgG and IgM retained by the column.

To assess whether the majority of the IgG and IgM in the eluate fraction (XNA⁺) were the result of non-specific binding to the column or were indeed galactosyl α(1, 3) galactose-reactive, natural antibodies (XNA) adsorption experiments were carried out using porcine erythrocytes. Porcine red blood cells (pRBCs) were chosen for this adsorption study as they have cell surface galactosyl α(1, 3) galactose moieties but lack Fc receptors which can non-specifically bind IgG. pRBCs were isolated from heparinized pig blood following processing with LSM (Organon Teknika, Durham, N.C.). The erythrocytes were taken from the base of the red cell pellet to avoid contamination with granulocytes or PBMC. The pRBCs were washed with HBSS and counted to determine cell number and purity. Natural antibodies (XNA) were isolated from pooled human serum by affinity chromatography as previously described (Galili, U. et al. (1987) Proc. Natl. Acad. Sci. USA 84:1369). The XNA fraction was diluted so that the IgM concentration was equal to that of the XNA⁺ sample. 2×10⁷, 2×10⁸, 2×10⁹, or 2×10¹⁰ pRBCs were incubated with 2 ml samples of XNA⁺ or XNA⁻ for 20 minutes at 4° C. with gentle rocking. The pRBCs were spun down at 1,500 rpm for 10 minutes at 4° C. The total IgG and IgM concentrations as well as the αGal/BSA reactivity of the supernatants were determined by ELISA for each of the adsorbed samples. The cc Gal/BSA reactivity and the IgG and IgM concentrations were determined pre- and post-adsorption. With 2×10¹ pRBC, the adsorbed XNA⁺ underwent a 92% reduction in αGal/BSA IgM reactivity and a 70% depletion of total IgM; for IgG, these values were 62% and 64% respectively. XNA⁻ was adsorbed with pRBCs in parallel; no reduction in the immunoglobulin concentration was observed. galactosyl α(1, 3) galactose-reactive natural antibody affinity purified under these conditions may contain as much as 30% non-specific IgM and much lower levels of contaminating IgG.

In order to determine if galactosyl α(1, 3) galactose-reactive natural antibodies comprise a constant percentage of total immunoglobulin, the immunoglobulin concentrations of the affinity purified galactosyl α(1, 3) galactose-reactive natural antibodies from ten serum samples were quantified by competitive ELISA. The concentrations of IgG natural antibodies ranged from 39 to 153 μg/ml with a mean of 65.3 μg/ml, while for IgM XNAs the concentrations ranged from 24 to 63 μg/ml with a mean of 40.1 μg/ml. Table 1 shows the percentage of IgG and IgM natural antibodies in different individuals.

TABLE 1
IgG

| Plasma Sample | Total IgG (μg/ml) | XNA + (IgG μg/ml) | Percent XNA + |
|---|---|---|---|
| 1 | 4100 | 56 | 1.4% |
| 2 | 6333 | 153 | 2.4% |
| 5 | 3225 | 39 | 1.2% |
| 6 | 4808 | 58 | 1.2% |
| 7 | 4117 | 72 | 1.7% |
| 8 | 4060 | 75 | 1.8% |
| 9 | 4200 | 46 | 1.1% |
| 10 | 4792 | 53 | 1.1% |
| 11 | 4708 | 47 | 1.0% |
| 14 | 3650 | 59 | 1.6% |
| 15 | 3250 | 51 | 1.6% |

IgM

| Plasma Sample | Total IgM (μg/ml) | XNA + (IgM μg/ml) | Percent XNA + |
|---|---|---|---|
| 1 | 305 | 17 | 5.6% |
| 2 | 1103 | 49 | 4.4% |
| 5 | 299 | 24 | 8.0% |
| 6 | 1033 | 44 | 4.3% |
| 7 | 742 | 39 | 5.3% |
| 8 | 1035 | 63 | 6.1% |
| 9 | 563 | 26 | 4.6% |
| 10 | 647 | 45 | 7.0% |
| 11 | 1070 | 42 | 3.9% |
| 14 | 810 | 37 | 4.6% |
| 15 | 672 | 32 | 4.8% |

To determine if the amount of galactosyl α(1, 3) galactose-reactive natural antibodies was related to total IgG or IgM, the immunoglobulin concentrations of the purified natural antibodies were compared with the levels of IgG and IgM in the original serum. The calculated percentage of galactosyl α(1, 3) galactose-reactive IgG ranged from 1.0 to 2.4% with a mean of 1.5% while for IgM it was 3.9 to 8.0% with a mean of 5.3%. Based upon this sample, the amount of natural antibodies in human serum does not appear to represent a constant percentage of total immunoglobulin. With each of the serum samples assessed, more IgG XNA⁺ was isolated than IgM XNA⁺; however, the overall percentage of XNA⁺ immunoglobulin relative to total immunoglobulin was substantially higher for IgM than for IgG.

Previously it has been estimated that galactosyl α(1, 3) galactose-reactive IgG accounts for approximately 1% of total IgG (Galili, U. et al. (1984) J. Exp. Med. 160:1519; Galili, U. et al. (1985) J. Exp. Med. 162:573); more recently, it has been reported that IgM natural antibody comprises 1-4% of total IgM (Parker, W. et al. (1994) J. Immunol. 153:3791). While the results presented are in agreement with these estimates, a substantially higher percentage of natural antibodies in some samples is reported here. The adsorption studies with porcine red blood cells (pRBC) suggest that there may be contaminating non-specific IgM in the affinity purified natural antibodies and consequently these values could be an overestimate by 30%. However, the non-galactosyl α(1, 3) galactose reactive IgM remaining after adsorption could also be due to a loss of function. Consistent with this, it has been observed that the natural antibodies undergo a substantial reduction in ability to bind galactosyl α(1, 3) galactose following acid elusion from protein A. As natural antibodies were eluted from the galactosyl α(1, 3) galactose column under acid conditions, the loss of galactosyl α(1, 3) galactose reactivity by some of the immunoglobulin in the eluate fraction would not be unexpected.

### Example 4

### Low Expression of Galactosyl α(1, 3) Galactose XNAS in Individuals with the Blood Group B Antigen

The inventors have made the surprising discovery that a moiety on human cells which is similar to the galactosyl α(1, 3) galactose moiety results in a significant reduction of anti-galactosyl α(1, 3) galactose natural antibody in human serum. Poly-N-acetyllactosamines in human erythrocytes carry the ABO-blood group antigens. In humans the terminal galactose is first substituted with α(,12) fucose, forming the H antigen. In B blood group individuals the H antigen is further modified, by an α(1,3)galactosyltransferase, by the addition of a(1,3)galactose. This α(1,3)galactosyltransferase is different from the α(1,3)galactosyltransferase present in New World primates and swine. It requires the presence of the fucosyl moiety on the H antigen. The swine α(1,3)galactosyltransferase does not require a fucose attached to N-acetyllactosamine. To detect possible antibody cross reactivity between the blood group B antigen and the galactosyl α(1, 3) galactose determinant, the relative level of galactosyl α(1, 3) galactose reactive natural antibodies in human serum from an equal number (n=12) of serum samples from the A, B, AB and O blood groups were assayed by direct ELISA. To ensure that the antibody binding detected was due entirely to the galactosyl α(1, 3) galactose determinant, each serum sample was also tested for BSA reactivity and serum samples exhibiting BSA reactivity (approximately 10% of the samples) were eliminated.

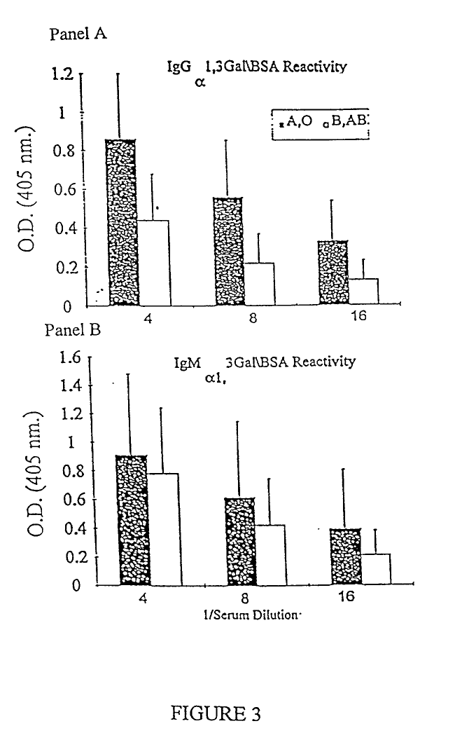

The overall galactosyl α(1, 3) galactose reactivity of the B antigen expressing blood groups (AB,B) when compared with the non-B antigen expressing blood groups (A,O) indicated a reduction in the level of natural antibody in the presence of the B antigen. See FIG. 3. This effect was more striking with IgG than with IgM. In order to quantify the observed reduction more precisely, a statistical analysis of the O.D. values for each of the serum dilutions was undertaken. The data was analyzed by comparing the non-B-expressing donors (A,O) against the B expressing donors (AB,B) using the Student's T Test. The results indicated that there was significant difference between the combined B and AB groups, when compared with the A and O blood groups for IgG (p<0.0002) and IgM (p<0.06) at the three (1/4, 1/8, 1/16) serum dilutions assayed. These results support the hypothesis that the human natural antibody population is regulated by the presence of the B antigen on human blood cells.

### Example 5

### Transient Effect of Galactosyl α(1, 3) Galactose Natural Antibody Depletion

In vitro experiments were performed to measure the recipient plasma natural antibody levels before and after galactosyl α(1, 3) galactose column perfusion. Following column perfusion, a porcine kidney was transplanted into a monkey without subsequent hyperacute rejection. In all of the cases (n=8), the level of natural antibodies in the monkey plasma decreased to background levels after column perfusion as measured by flow cytometric analysis (i.e., measuring porcine reactivity) and ELISA (measuring galactosyl α(1, 3) galactose reactivity). However, the natural antibodies levels rebounded several days later. As demonstrated for one of the longest xenograft survivors, the level of natural antibodies in the plasma remained low for only a short time. By day 15, the galactosyl α(1, 3) galactose reactive natural antibody returned to pre-adsorption levels of IgM while the IgG level rose to ten-fold its original value. These results not only emphasize the importance of depleting the galactosyl α(1, 3) galactose reactive natural antibody but also demonstrates the importance of regulating the natural antibody producing B cell population in xenotransplantation.

### Example 6

### Production of a Retroviral Vector for Delivery of α(1,3)galactosyltransferase to Cells

A 1145 bp EcoRI-Cac8I restriction cDNA fragment containing the coding region of porcine α(,13)galactosyltransferase (α1,3GT) from pSa13GT1 (Strahan, K. M. et al. (1995) Immunogenetics 41:101) was cloned into pBluescript II KS (−) (Stratagene) and then the murine phosphoglycerate kinase (PGK) transcriptional promoter was inserted upstream of the α(1,3)galactosyltransferase coding region to construct PGKα1,3GT. PGKα1,3GT is then introduced into the 3′ LTR of the retrovirus vector N2A (Armentano, D. et al. (1987) J. Virol. 61:1647; Bordignon, C. et al. (1989) Proc. Natl. Acad. Sci. USA 86:6784; Hayashi, H. et al. (1995) Transplant. Proc. 27:179) to construct a provirus carrying α1,3GT driven by the PGK transcriptional promoter (PGK α1,3GTRV). PGKα1,3GTRV is then introduced into the amphotropic retroviral packaging cell line PA317 (Miller, A. D. and Buttimore, C. (1986) Mol. Cell. Biol 6:2895) by transfection to create a virus producer cell line. Following selection of the transfected clones with G418, producer clones are picked and expanded to test viral titer. Cell lines producing high titer helper-free virus are used to prepare retrovirus stocks (α1,3GTRV).

To test whether α1,3 GTRV can transfer functional α1,3 GT capable of generating galactosyl α(1, 3) galactose epitopes, COS cells (α1,3Gal negative) are infected with the recombinant virus (M.O.I >2) and it is determined whether the α1,3Gal epitope is expressed on the cell surface proteins by staining with the lectin from Bandeiraea simplicifolia (IB₄, BS-I isolectin B₄) which specifically recognizes the galactosyl α(1, 3) galactose epitope for the α1,3Gal as well as purified galactosyl α(1, 3) galactose reactive human natural antibodies and analyzed by flow cytometry using standard methodologies. The levels of galactosyl α(1, 3) galactose epitopes encoded for by the transduced enzyme are compared to the level normally present on cells from swine.

To test the efficacy of transduction with α1,3GTRV, RAG-1 (R⁻) deficient mice which lack mature B and T cells (Mombaerts, P. et al. (1992) Cell 68:869) are reconstituted with bone marrow from α1,3GT deficient mice (A⁻) mice transduced by an α(1,3GTRV or a control retrovirus ENJ36 (Fraser, C. C. et al. (1995) J. Immunol. 154:1587) carrying only the NEO resistance gene. M.O.I are greater than 2 for all infections and performed as described previously (Sykes, M. et al. (1993) Transplantation 55:197). On day 14 post reconstitution, the mice are sacrificed and colony forming units from the spleen (CFU-S) are harvested. Cell suspensions are prepared and stained with IB₄ lectin or purified galactosyl α(1, 3) galactose binding human natural antibody and analyzed by flow cytometry. DNA is prepared from 1/2 of each colony and analyzed by PCR to determine whether the colony was derived from A-bone marrow.

### Example 7

### A Murine Model for the Induction of Tolerance to Cells Expressing Galactosyl α(1, 3) Galactose Moiety

The murine system described herein can be used to evaluate a particular component, e.g., a sequence which encodes an galactosyl α(1, 3) galactose moiety forming enzyme, for the ability to promote tolerance to the galactosyl α(1, 3) galactose moiety. To test the ability of retrovirally transfected cells to induce tolerance to the galactosyl α(1, 3) galactose epitope, a murine host which lacks both (a) galactosyl α(1, 3) galactose epitopes that cause deletion of developing B cells producing galactosyl α(1, 3) galactose reactive antibodies, and (b) galactosyl α(1, 3) galactose reactive natural antibody capable of rejecting the modified bone marrow cells was developed. α1,3GT deficient mice (A⁻) are crossed with RAG-I (R⁻) deficient mice which have been previously shown to lack mature B and T cells (Mombaerts, P. et al. (1992) Cell 68:869) to generate α1,3GT−/−, RAG-1−/− (A⁻R⁻) mice. In order to construct A⁻R⁻ mice, homozygous A⁻ and R⁻ deficient mice are crossed. The resulting F1 generation is then intercrossed to generate A⁻R⁻mice at an expected frequency of 1 in 16. Offspring are genotyped based on Southern hybridization. The A⁻R⁻ are then be intercrossed to establish a colony. Irradiated A⁻R⁻ mice are reconstituted with bone marrow cells from A⁻ mice transduced with retrovirus carrying α1,3GT, or a control retrovirus carrying only the neomycin resistance gene. The ability of reconstituted mice to produce galactosyl α(1, 3) galactose reactive antibodies is used to measure whether gene therapy leads to B cell tolerance in this model.

### Example 8

### Generation of Porcine Anti-Human Anti-Idiotypic Antibodies

An galactosyl α(1, 3) galactose carbohydrate column was used to isolate XNA⁺ (250 μg) from the serum of a single donor which was emulsified with Complete Freund's Adjuvant (CFA) and used to immunize a miniature swine. This animal was repeatedly boosted with purified natural antibodies until a high level of reactivity against human immunoglobulin was produced. After the fifth boost, the animal was exsanguinated and serum was collected and stored at −20° C. To remove non-idiotype specific anti-human antibodies, the sera was passed over a column to which natural antibody depleted human Ig had been conjugated. The flowthrough fractions were then screened for differential binding to XNA⁺ or XNA⁻ coated plated by ELISA. The partially purified flowthrough reacted preferentially with XNA⁺ suggesting that a pig anti-idiotypic reagent was generated which was able to distinguish XNA⁺ and the XNA⁻ fractions of the original donor.

### Example 9

### Human Natural Antibody from Unrelated Donors Express a Crossreactive Idiotype

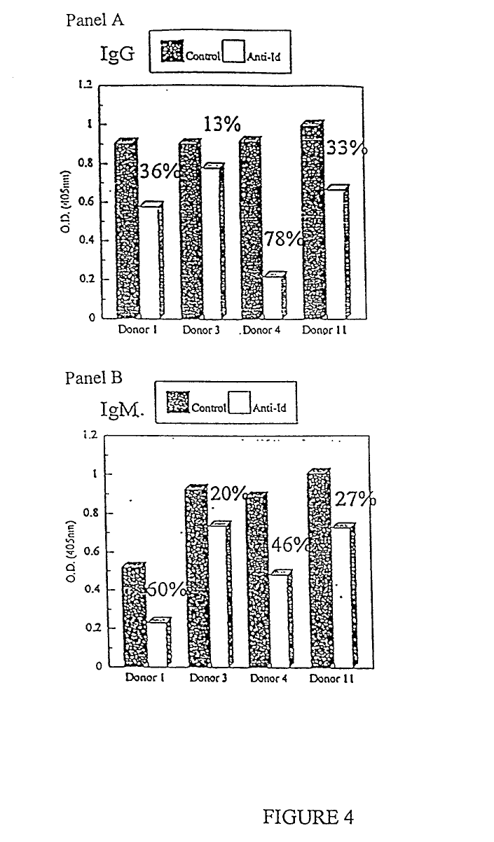

The ability of the anti-idiotype antisera produced in the example above to inhibit serum natural antibody binding to galactosyl α(1, 3) galactose was assessed using a competitive ELISA in which sera from four unrelated donors were tested. The binding of natural antibody to α1,3Gal/BSA coated plates was inhibited 20-60% for IgM and 13-78% for IgG. See FIG. 4. These results show that a crossreactive natural antibody idiotype is expressed in unrelated individuals. Such cross reactivity is indicative of structural relatedness possibly as a result of limited V gene usage in the human natural antibody population.

### Example 10

### Production of Mouse Anti-Human Anti-Idiotypic Antibodies

The cross reactivity of the porcine anti-idiotype reagent with natural antibody from four unrelated individuals indicates the practicality of producing monoclonal anti-idiotype reagents. Anti-idiotype reagents are useful for therapeutic applications and for screening natural antibody producing EBV transformed B cells for the predominating idiotype.

Mouse anti-human anti-idiotypic monoclonal antibody producing hybridomas can be generated against human natural antibody that is affinity purified from plasma or from EBV transformed clonal B-cells. To purify a human antibody fraction from all other serum components, an initial affinity purification step is performed using an anti-human IgG and IgM column. The purified Ig is then subjected to an galactosyl α(1, 3) galactose column to enrich for galactosyl α(1, 3) galactose reactive natural antibodies (XNA⁺). The anti-galactosyl α(1, 3) galactose antibody depleted fraction (XNA⁻) is also collected. The affinity purified XNA⁺ is then concentrated in a Centricon column (Amicon) and quantitated for total IgG and IgM concentrations by competitive ELISA. Purified XNA⁺ and XNA⁻ immunoglobulin are subjected to SDS-PAGE to ensure purity. This procedure provides material for experiments including 2-Dimensional Electrophoresis and the production of anti-idiotypic antibodies.

Mice are immunized and after testing for strong anti-human Ig reactivity, the mice are sacrificed and spleen cells fused. Hybridoma supernatant is screened directly by ELISA on XNA⁺ and XNA⁻ coated plates. Wells producing reactivity against the galactosyl α(1, 3) galactose-specific XNA⁺ coated plates, but not to the XNA⁻ coated plates, are selected for further analysis. These hybridoma cell lines are expanded and cryopreserved. Putative anti-idiotypic monoclonals are also tested for their ability to block natural antibody binding to galactosyl α(1, 3) galactose coated ELISA plates.

### Example 11

### Elimination of Anti-α(1-3)galactose α(1-3)gal) Reactive Antibodies by Gene Therapy

Retroviral gene therapy can be used in a tolerance inducing regimen to eliminate production of galactosyl α(1,3)galactose reactive antibodies. This method will inhibit graft rejection mediated by α(1-3)Gal reactive antibodies which is important in xenotransplantation across discordant barriers. α1,3GT mice knockout mice (GT⁰) (Thall et al., 1995, J. Biol. Chem. 270:21437) capable of producing galactosyl α(1,3)galactose reactive antibodies (Thall et al., 1996, Transplantation Proceedings 28:561) were used as a model system for introducing porcine α1,3GT, by retrovirus mediated gene transfer, into bone marrow lymphohematopoietic progenitors on production of galactosyl α(1,3)galactose reactive antibodies. The reconstitution of lethally irradiated GT⁰ mice with porcine α1,3GT transduced syngeneic bone marrow effectively prevents the development of galactosyl α(1,3)galactose producing B cells.

Retrovirus Vectors Capable of Transferring Porcine α(1-3)GT as a Constitutively Expressed Gene into Bone Marrow Derived Cells.

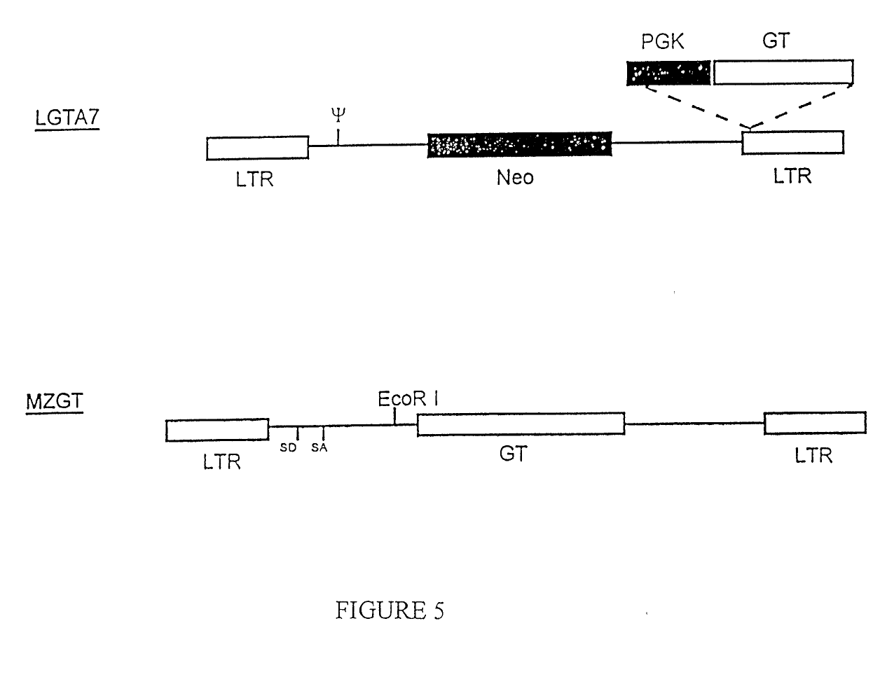

Two retroviral vectors carrying the gene encoding porcine αGT was constructed, see FIG. 5. The first vector (LGTA7) is a N2A (Hantzapoulous et al., 1989, PNAS 86:3519) based retroviral vector in which αGT expression is driven by the murine phosphoglycerate kinase (PGK) promoter. The second vector (MZGT) is a Macrozen (Johnson et al., 1989, EMBO J. 8:441) based vector in which expression of α1,3GT is driven by the myeloproliferative sarcoma virus promoter contained in the 5′LTR of the virus. Two different retroviral vector were designed because it is possible that different vectors may be more or less effective in allowing α1,3GT expression. In order to derive virus producer cell lines, the above constructs were introduced separately into AM12 amphotropic packaging cell lines (Markovitz et al., 1988, Virology 167:400) and virus producing lines established as described previously (Fraser et al., 1995, J. Immunol. 154:1587). Amphotoropic packaging cells were used in a preclinical primate xenotransplantation mode.

To test whether the recombinant retroviruses were able to transfer functional α1,3GT expression, Vero cells (African Green monkey kidney epithelial cell lines, α1,3GT negative) were transduced with LGTA7 or MZGT virus produced in AM12 cells. Surface expression of galactosyl α(1,13)galactose epitopes was then analyzed on selected clones by staining with FITC labeled lectin from Bandeiraea simplicifolia (BS=I isolectin B₄) specific for galactosyl α(1,3)galactose and analyzed by flow cytometry. In all experiments, Vero cells infected with a control retrovirus containing only the neomycin resistance gene (NEO) were analyzed in parallel. Vero cells infected with LGTA7 expressed galactosyl α(1,3)galactose epitopes on the cell surface at levels detectable by flow cytometry. Surface expression of galactosyl α(1,3)galactose epitopes was stable and could be detected on the surface of clones after several months in culture. No surface expression of galactosyl α(1,3)galactose epitopes was detected on control NEO transduced cells. In order to confirm that the staining with IB4 lectin was indeed a consequence of galactosyl α(1,3)galactose epitope expression encoded for by the introduced transgene, Vero cells transduced with LGTA7 were treated with α-galactosidase. Treatment of LGTA7 transduced Vero cell clones with α-galactosidase specifically reduced galactosyl α(1,3)galactose expression detectable by staining IB₄-FITC. Similar results were obtained using MZGT amphotropic retrovirus. These data indicate that LGTA7 and MZGT retroviruses are able to transfer expression of porcine α1,3GT, which in turn catalyzes the addition of galactose epitopes in a α(1-3) linkage on the surface of primate cells.

Expression of Retrovirally Transduced Porcine αGT in Bone Marrow Derived Cells from GT⁰ Mice can Eliminate Production of α(1-3)Gal Reactive Antibodies

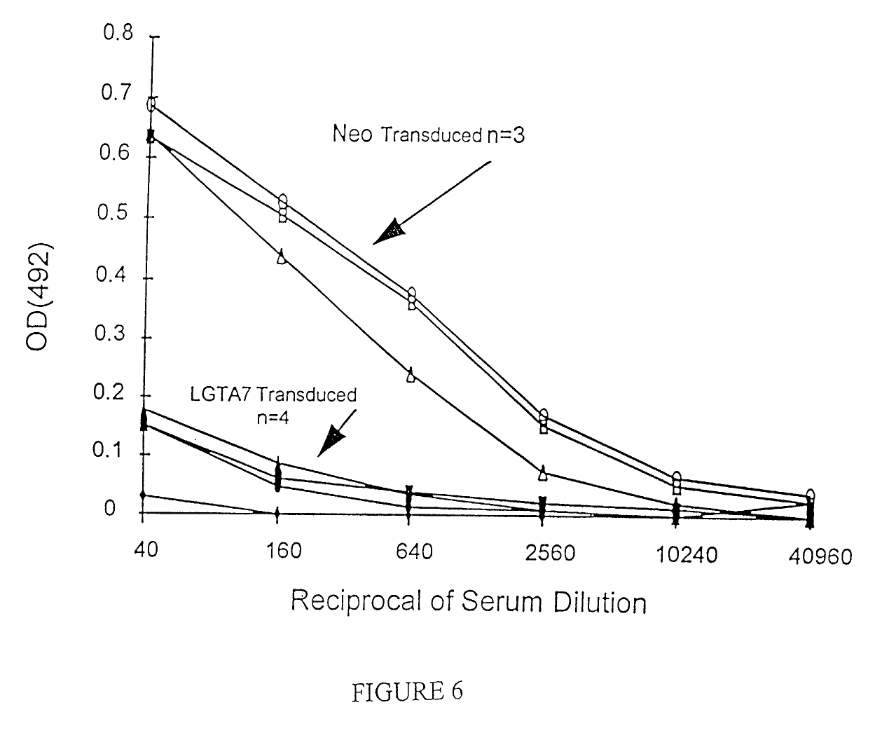

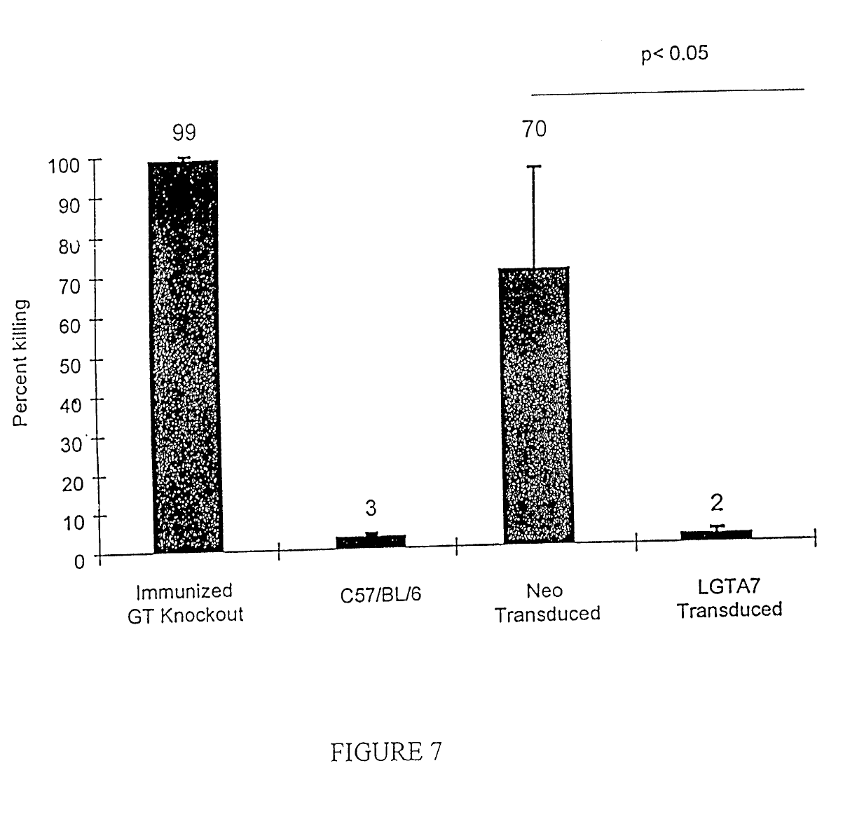

Bone marrow cells from GT⁰ mice treated in vivo for 7 days prior to harvest with 150 mg/kg 5-fluorouracil were transduced by co-cultivation with the LGTA7 virus producer cells or control lines producing virus containing only the neomycin resistance gene as described (Fraser et al., 1995, J. Immunol. 154:1587). After 4 days of in vitro culture, transduced bone marrow cells were harvested and lethally irradiated (10.25Gy) GT⁰ mice were reconstituted with 10⁶ LGTA7 (group 1, n=4) or Neo transduced (group 2, n=3) bone marrow cells. Starting at 3 weeks post bone marrow transplantation mice in each group were bled and the presence of α(1-3)Gal reactive serum antibodies analyzed by ELISA. As shown in FIG. 6, while galactosyl α(1,3)galactose reactive serum antibodies were readily detectable in control mice reconstituted with Neo transduced bone marrow, mice reconstituted with LGTA7 transduced bone marrow failed to develop α(1-3)gal reactive antibodies. Serum galactosyl α(1,3)galactose reactive antibodies were undetectable in mice reconstituted with LGTA7 transduced bone marrow analyzed for at least 18 weeks post bone marrow transplantation. To confirm the results obtained by ELISA, serum from mice in each group was analyzed for the presence of antibodies capable of lysing α(1-3)Gal positive porcine PK-15 cells in the presence of rabbit complement as described (Koren et al., 1994, Transplantation Proceedings 26:1166; and Koren et al., 1994, Transplantation Proceedings 26:1336). As show in FIG. 7 while GT⁰ mice immunized with porcine PBMC, and mice reconstituted with Neo transduced bone marrow contained serum antibodies capable of mediating lysis of PK-15 cells, serum antibodies were not detectable in normal mice immunized with porcine PBMC or mice reconstituted with LGTA7 transduced bone marrow capable of mediating lysis of PK-15 cells. Together, these data demonstrate that reconstitution of lethally irradiated GT⁰ mice with porcine α1,3GT transduced syngeneic bone marrow effectively prevents the development of galactosyl α(1-3)galactose producing B cells.

### Example 12

### Induction of Tolerance to Galactosyl α(1,3) Galactose Moieties

The following procedure was designed to induce tolerance to a galactosyl α(1, 3) galactose moiety in a human or Old World primate. It can be used to prepare an Old World primate, a baboon (Papio anubis), for receipt of a kidney from a miniature swine donor.

The procedure is designed to reduce the anti-galactosyl α(1, 3) galactose natural antibody (XNA) response of the recipient, by introducing autologous stem cells which present galactosyl α(1, 3) galactose moieties into the recipient. Recipient bone marrow is aspirated from the iliac crest of the recipient. This provides autologous cells for the production of a feeder layer which will be used to culture recipient stem cells. Stromal cell cultures are generated by separating low density bone marrow cells over a Ficoll gradient and plating 2×10⁶ cells per well in a 24-well plate pre-coated with 1% gelatin. The cultures are incubated, in 5% CO₂ and 95% humidity, using M199 medium containing 10% fetal bovine serum, 10% horse serum and 10⁻⁶ M hydrocortisone at 37° C. for one week, and then, at 33° C. for two additional weeks. Medium is demi-depleted at weekly intervals and supplemented with fresh medium for 3 weeks while a confluent stromal cell layer is formed.

After preparation of the feeder layer, a second bone marrow aspiration is performed to provide autologous stem cells for transduction. Transduction of CD34⁺ autologous bone marrow cells is performed in the presence of the preformed stromal cell culture. CD34⁺ cells are enriched from the low density Ficoll gradient fractions of recipient bone marrow harvested 3 days (day −3) prior to bone marrow transplantation, by immunoadsorption using a Ceprate column (CellPro Inc., Bothel, Wash.). The bone marrow cells are then plated at 5×10⁴ cells/ml/well onto the autologous stromal cell layer described above. The CD34⁺ cells are cultured overnight in M199, 10% FBS, 10% horse serum supplemented with 100 ng/ml rhSCF (R & D Systems, Minneapolis, Minn.), 100 ng/ml rhIL-3 (Sandoz Pharmaceuticals Co., Basel, Switzerland) at 37° C. Cultures are exposed to α(1,3)GT expressing retroviral supernatant (4×10⁶ infectious particles/ml of amphotropic recombinant virus) for 18 hr. in the presence of 6 μg/ml of polybrene and growth factors. The cells are reexposed to cytokines and virus for a second time following the above procedure. (A control transduction experiment is set up under identical conditions except that the cells do not receive retrovirus.) On day 0, transduced adherent and non-adherent populations are harvested and infused into the same animal from which the marrow is harvested.

A non-myeloablative conditioning regimen is used to prepare the recipient for transplantation of the engineered autologous stem cells. The recipient receives nonlethal total body irradiation of 300 Rads from a ⁶⁰Co source on day −3. The animal is further treated with thymic irradiation of 700 Rads on day −1, and anti-thymocyte globulin (ATG, Upjohn, Kalamazoo, Mich.), 50 mg/kg, i.v., on days −3, −2, and −1.

Prior to introduction of the galactosyl α(1, 3) galactose expressing recipient cells natural antibodies are removed from the recipient's circulation by passing the recipient's blood through a galactosyl α(1, 3) galactose affinity column. The galactosyl α(1, 3) galactose affinity column is prepared according to the manufacturer's directions (Alberta Research Council, Edmonton, Calif.). The recipient is anesthetized with halothane and maintained by general endotracheal intubation anesthesia with monitoring of blood pressure, blood oxygen saturation, blood gases and pH throughout the case. In addition to an internal jugular vein cutdown, a brachial artery indwelling catheter is placed to allow for direct blood pressure measurements. A splenectomy may be performed on the recipient. The recipient's aorta and vena cava are then exposed and cannulated using silastic shunts. The aortic cannula is connected either to the column inlet, and the circuit is completed by connecting the recipient vena cava cannula to the column outlet. Flow rates are measured using a volume meter. Continuous monitoring by an anesthesiologist is required to maintain euvolemia and manage introperative coagulopathy, anemia, and hypothermia. The recipient's blood is perfused through the column for sixty minutes. The efficacy of the perfusion technique for the removal of natural antibodies is assayed by flow cytometric analysis.

Administration of cyclosporine (Sandoz Pharmaceuticals Co., Basel, Switzerland) is given between days 0 and day 28, at a dose of 15 mg/kg/day, i.v., to maintain a plasma level of greater than 300 ng/ml. Recombinant human GM-CSF (Sandoz Pharmaceuticals Co., Basel, Switzerland) is given subcutaneously from days 0 through 14 at a dose of 5 μg/kg/day. Ofloxacin is given throughout the neutropenic period as prophylaxis against infection, starting on day −3, at a dose of 50 mg/day i.v.

After depletion of anti-galactosyl α(1, 3) galactose natural antibodies from the recipient's blood, the engineered recipient cells are introduced into the recipient. The recipient is then monitored for the production of galactosyl α(1, 3) galactose antibodies. After establishing that the galactosyl α(1, 3) galactose antibodies have decreased or been eliminated the porcine bone marrow stem cells and kidney can be transplanted into the recipient as is described in Example 12 below.

Human natural antibodies can be detected with the following ELISA assay. Nunc Maxisorb plates are coated with 100 μl/well of 5 μg/mL of βGal (1→3) βGal (1→4) βGlc-X-Y conjugated to BSA (Alberta Research Council, Canada) in carbonate bi-carbonate buffer (pH>9.5). These plates are then incubated at 4° C. for overnight. Coated plates are washed 5-6 times with PBS-Tween-20 (0.5%) and blocked with 200 μL/well of 1% BSA (Sigma, MO) in PBS-Tween-20 (0.5%). For 1 hour at 37° C. The plates are either used immediately or frozen at −20° C. until used. Before use, the plates are washed 5-6 times with PBS-Tween-20 (0.5%) and loaded with 100 μl/well of graded doses (0.016%-2%) of baboon or human serum. The plates are then incubated for 1 hour at 37° C. and washed 5-6 times with PBS-Tween-20 (0.5%). Bound antibodies are detected using polyclonal donkey anti-human IgG (Accurate, NY) and rabbit anti-human igM (Dako, Denmark) conjugated to Horseradish peroxidase (HRP). The plates are incubated for 1 hour at 37° C. After the plates are washed 5-6 times with PBS-Tween-20 (0.5%), color development is achieved by using o-phenylenediamine dihydrochloride (OPD, sigma, MO) as a substrate at 0.9 mg/mL in phosphate citrate buffer with urea hydrogen peroxidase (Sigma, MO). After 13 minutes of incubation at room temperature and in complete darkness, the plates are blocked with 50 μL of 2N H₂SO₄ and absorbance at 490 nm is measured by THERMOmax plate reader (Molecular Devices, CA).

Mouse natural antibodies reactive with Gal α1.3 Gal can be detected in an assay identical to the above-described assay except for the use of donkey anti-mouse IgG and donkey anti-mouse IgM (Acurate, NY) as the detecting antibodies.

### Example 13

### Induction of Tolerance to a Graft Which Presents Galactosyl α(1,3) Galactose Moieties

The following procedure was designed to lengthen the time an implanted tissue which displays a galactosyl α(1, 3) galactose moiety survives in a human or Old World primate prior to rejection. The tissue can be, e.g., hematopoietic stem cells, or an organ, e.g., a liver, a kidney, or a heart. The main strategies are: the elimination of natural antibodies which recognize the galactosyl α(1, 3) galactose moiety on the graft by implantation in the recipient of galactosyl α(1, 3) galactose presenting recipient stem cells (as described in Example 11 above); the reduction of recipient anti-donor T and NK cell activity; the transplantation of tolerance-inducing donor bone marrow; and the administration of a short course of a help reducing agent at about the time of introduction of the graft.

Elimination of the Anti-Galactosyl α(1,3) Galactose Natural Antibody Response.

Recipient natural antibodies which recognize the galactosyl α(1, 3) galactose moiety are minimized as described in Example 11 above.

Preparation of the Recipient for Donor Stem Cells.

Recipient T and NK cell activity is inactivated by the administration of anti-T and anti-NK cell antibodies. Thus, on the third, second and first day prior to introduction of donor stem cells, a commercial preparation (Upjohn, Kalamazoo, Mich.) of horse anti-human anti-thymocyte globulin (ATG) is injected into the recipient ATG eliminates mature T cells and NK cells that could promote would otherwise cause rejection of the bone marrow cells used to induce tolerance. The recipient is anesthetized, an IV catheter is inserted into the recipient, and 6 ml of heparinized whole blood are removed before injection. The ATG preparation is then injected (50 mg/kg) intravenously. Six ml samples of heparinized whole blood are drawn for testing at time points of 30 min., 24 hours and 48 hours. Blood samples are analyzed for the effect of antibody treatment on NK cell activity (testing on K562 targets) and by flow cytometric analysis for lymphocyte subpopulations, including CD4, CD8, CD3, CD11b, and CD16. If mature T cells and NK cells are not sufficiently inhibited, ATG can be re-administered at later times in the procedure, both before and after organ transplantation. Anti-human ATG obtained from any mammalian host can also be used, e.g., ATG produced in pigs, although thus far preparations of pig ATG have been of lower titer than horse-derived ATG. ATG is superior to anti-NK monoclonal antibodies, as the latter are generally not lytic to all host NK cells, while the polyclonal mixture in ATG is capable of lysing all host NK cells. Anti-NK monoclonal antibodies can, however, be used.

The presence of donor antigen in the host thymus during the time when host T cells are regenerating post-transplant is critical for tolerizing host T cells. If donor hematopoietic stem cells are not able to become established in the host thymus and induce tolerance before host T cells regenerate repeated doses of anti-recipient T cell antibodies may be necessary throughout the non-myeloablative regimen. Continuous depletion of host T cells may be required for several weeks.

Sublethal irradiation is administered to the recipient between days −3 and −1 prior to donor stem cell transplantation to create hematopoietic space. Sublethal whole body irradiation is sufficient to permit engraftment with minimal toxic effects to the recipient. Whole body radiation (300 Rads) can be administered to nonhuman primate recipients from a bilateral cobalt teletherapy unit at 10 Rads/min.

The creation of thymic space is also useful in the induction of tolerance. Local irradiation of the thymus (700 Rads) can be used to induce thymic space. Thymic irradiation can be administered on the day prior to donor stem cell administration.

Administration of Porcine Donor Stem Cells.

To promote long-term survival of the implanted organ through T-cell and B-cell mediated tolerance, donor bone marrow cells are injected into the recipient to form chimeric bone marrow. (As liver is the major site of hematopoiesis in the fetus, fetal liver can also serve as an alternative to bone marrow as a source of hematopoietic stem cells.) Donor bone marrow cells home to appropriate sites of the recipient and grow contiguously with remaining host cells and proliferate, forming a chimeric lymphohematopoietic population. The presence of donor antigens in the bone marrow allows newly developing B cells, and newly sensitized T cells, to recognize antigens of the donor as self, and thereby induces tolerance for the implanted organ from the donor. To stabilize the donor BMC, porcine IL-3 and stem cell factor (BioTransplant, Inc. Charlestown, Mass.) can be administered to the recipient between days 0 through 14 at 10 μg/kg/day. Bone marrow can be harvested and injected intravenously (7.5×10⁸/kg) as previously described (Pennington et al., 1988, Transplantation 45:21-26). Should natural antibodies be found to recur before tolerance is induced, and should these antibodies cause damage to the graft, the protocol can be modified to permit sufficient time following BMT for humoral tolerance to be established prior to organ grafting.

To follow chimerism, two color flow cytometry can be used. This assay uses monoclonal antibodies to distinguish between donor class I major histocompatibility antigens and leukocyte common antigens versus recipient class I major histocompatibility antigens. Chimerism can also be followed by using quantitative polymerase chain reaction to amplify porcine specific sequences, thereby indicating the presence of porcine cells.

Cyclosporine (Sandoz Pharmaceuticals Co., Basel, Switzerland) is administered for about 28 days, beginning at the time of donor cell implantation (or a few days before), at a dose of 15 mg/kg/day, i.v., to maintain a plasma level of greater than 300 ng/ml.

Introduction of the Porcine Graft.

After donor stem cells have been administered a miniature swine kidney is implanted into the recipient. When an organ graft is placed in such a recipient several months after bone marrow chimerism has been induced, natural antibody against the donor will have disappeared, and the graft should be accepted by both the humoral and the cellular arms of the immune system. Organ transplantation can be performed sufficiently long following transplant of hematopoietic cells, that normal health and immunocompetence will have been restored at the time of organ transplantation. The use of xenogeneic donors allows the possibility of using bone marrow cells and organs from the same animal, or from genetically matched animals.

The approaches described above are designed to synergistically prevent the problem of transplant rejection. While any of these procedures may aid the survival of an implanted organ, best results are achieved when all steps are used in combination.

The method of introducing stem cells may be altered, particularly by (1) increasing the time interval between injecting hematopoietic stem cells and implanting the graft; (2) increasing or decreasing the amount of hematopoietic stem cells injected; (3) varying the number of hematopoietic stem cell injections; (4) varying the method of delivery of hematopoietic stem cells; (5) varying the tissue source of hematopoietic stem cells, e.g., a fetal liver cell suspension may be used; or (6) varying the donor source of hematopoietic stem cells. Although hematopoietic stem cells derived from the graft donor are preferable, hematopoietic stem cells may be obtained from other individuals, preferably from inbred donor strains, or from in vitro cell culture.

Irradiation of the recipient may make use of: (1) varying the absorbed dose of whole body radiation below the sublethal range; (2) targeting different body parts (e.g., thymus, spleen); (3) varying the rate of irradiation (e.g., 10 Rads/min., 15 Rads/min.); or (4) varying the time interval between irradiation and transplant of hematopoietic stem cells; any time interval between 1 and 14 days can be used, and certain advantages may flow from use of a time interval of 4-7 days.

Antibodies introduced prior to hematopoietic cell transplant may be varied by: (1) using monoclonal antibodies to T cell subsets or NK cells (e.g., anti-NKH1_{A}, as described by U.S. Pat. No. 4,772,552 to Hercend, et al., hereby incorporated by reference); (2) preparing anti-human ATG in other mammalian hosts (e.g., monkey, pig, rabbit, dog); or (3) using anti-monkey ATG prepared in any of the above mentioned hosts.

### Other Embodiments

The preferred tolerogen for use in methods of the invention is the galactosyl α(1, 3) galactose moiety. However, as is shown in Example 4 above, other moieties, e.g., the blood group B antigen, can induce a degree of tolerance to the galactosyl α(1, 3) galactose moiety. Thus, any moiety, particularly other carbohydrate moieties, which are sufficiently similar in structure to induce tolerance to the galactosyl α(1, 3) galactose moiety can be used in the methods and compositions described herein. Compounds can be screened for use as a tolerogen by testing for cross reactivity with anti-galactosyl α(1, 3) galactose antibodies. The ability of a candidate compound to bind the antibody is indicative of usefulness as a tolerogen.

The methods of the invention can also be used to induce tolerance to other natural antibody antigens, e.g., other carbohydrates which are the target of natural antibodies. The moiety to which tolerance is induced can be found as follows. Human natural antibodies can be isolated and depleted of galactosyl α(1, 3) galactose moiety reactive antibodies. The remaining natural antibodies can be tested against a panel of antigens, e.g., a panel of carbohydrate moieties, to select antigens for use in tolerization. Once the antigen is identified, tolerance can be induced by modifying the methods described herein for use with the new antigen.

The methods of the invention are particularly useful for replacing a tissue or organ afflicted with a neoplastic disorder, particularly a disorder which is resistant to normal modes of therapy, e.g., chemotherapy or radiation therapy. In preferred embodiments: the graft includes tissue from the digestive tract or gut, e.g., tissue from the stomach, or bowel tissue, e.g., small intestine, large intestine, or colon; the graft replaces a portion of the recipient's digestive system e.g., all or part of any of the digestive tract or gut, e.g., the stomach, bowel, e.g., small intestine, large intestine, or colon.

As is discussed herein, it is often desirable to expose a graft recipient to irradiation in order to promote the development of mixed chimerism. Mixed chimerism can be induced with less radiation toxicity by fractionating the radiation dose, i.e., by delivering the radiation in two or more exposures or sessions. Accordingly, in any method of the invention calling for the irradiation of a recipient, e.g., a primate, e.g., a human, recipient, of a xenograft the radiation can either be delivered in a single exposure, or more preferably, can be fractionated into two or more exposures or sessions. The sum of the fractionated dosages is preferably equal, e.g., in Rads or Gy, to the radiation dosage which can result in mixed chimerism when given in a single exposure. The fractions are preferably approximately equal in dosage. For example, a single dose of 700 Rads can be replaced with, e.g., two fractions of 350 Rads, or seven fractions of 100 Rads. Hyperfractionation of the radiation dose can also be used in methods of the invention. The fractions can be delivered on the same day, or can be separated by intervals of one, two, three, four, five, or more days. Whole body irradiation, thymic irradiation, or both, can be fractionated.

Methods of the invention can include recipient splenectomy.

As is discussed herein, contacting the recipient's blood with galactosyl (α1, 3) galactose epitopes can be used to deplete the host of natural antibodies. Other methods for depleting or otherwise inactivating natural antibodies can be used with any of the methods described herein. For example, drugs which inactivate natural antibodies, e.g., deoxyspergualin (DSG) (Bristol), or anti-IgM antibodies, can be administered to the recipient of an allograft or a xenograft. One or more of, DSG (or similar drugs), anti-IgM antibodies, and hemoperfusion, can be used to inactivate recipient natural antibodies in methods of the invention. DSG at a concentration of 6 mg/kg/day, i.v., has been found useful in suppressing natural antibody function in pig to cynomolgus kidney transplants.

Methods for the inactivation of thymic T cells or thymocytes are also included in embodiments of the invention. Some of the methods described herein include the administration of thymic irradiation to inactivate host thymic-T cells or to otherwise diminish the host's thymic-T cell mediated responses to donor antigens. It has been discovered that the thymic irradiation called for in xenogeneic methods of the invention can be supplemented with, or replaced by, other treatments which diminish (e.g., by depleting thymic-T cells and/or down modulating one or more of the T cell receptor (TCR), CD4 co-receptor, or CD8 co-receptor) the host's thymic-T cell mediated response. For example, thymic irradiation can be supplemented with, or replaced by, anti-T cell antibodies (e.g., anti-CD4 and/or anti-CD8 monoclonal antibodies) administered a sufficient number of times, in sufficient dosage, for a sufficient period of time, to diminish the host's thymic-T cell mediated response.

For best results, anti-T cell antibodies should be administered repeatedly. E.g., anti-T cell antibodies can be administered one, two, three, or more times prior to donor bone marrow transplantation. Typically, a pre-bone marrow transplantation dose of antibodies will be given to the patient about 5 days prior to bone marrow transplantation. Additional, earlier doses 6, 7, or 8 days prior to bone marrow transplantation can also be given. It may be desirable to administer a first treatment then to repeat pre-bone marrow administrations every 1-5 days until the patient shows excess antibodies in the serum and about 99% depletion of peripheral T cells and then to perform the bone marrow transplantation. Anti-T cell antibodies can also be administered one, two, three, or more times after donor bone marrow transplantation. Typically, a post-bone marrow transplant treatment will be given about 2-14 days after bone marrow transplantation. The post bone marrow administration can be repeated as many times as needed. If more than one administration is given the administrations can be spaced about 1 week apart. Additional doses can be given if the patient appears to undergo early or unwanted T cell recovery. Preferably, anti-T cell antibodies are administered at least once (and preferably two, three, or more times) prior to donor bone marrow transplantation and at least once (and preferably two, three, or more times) after donor bone marrow transplantation.

Some of the methods herein include the administration of hematopoietic stem cells (engineered autologous cells or donor cells) to a recipient. In many of those methods, hematopoietic stem cells are administered prior to or at the time of the implantation of a graft, the primary purpose of the administration of hematopoietic stem cells being the induction of tolerance to the graft. The inventors have found that one or more subsequent administrations (e.g., a second, third, fourth, fifth, or further subsequent administration) of hematopoietic stem cells can be desirable in the creation and/or maintenance of tolerance. Thus, the invention also includes methods in which hematopoietic stem cells are administered to a recipient, e.g., a primate, e.g., a human, which has previously been administered hematopoietic stem cells as part of any of the methods referred to herein.

While not wishing to be bound by theory, it is believed that repeated stem cell administration may promote chimerism and possibly long-term deletional tolerance in graft recipients. Accordingly, any method referred to herein which includes the administration of hematopoietic stem cells can further include multiple administrations of stem cells. In preferred embodiments: a first and a second administration of stem cells are provided prior to the implantation of a graft; a first administration of stem cells is provided prior to the implantation of a graft and a second administration of stem cells is provided at the time of implantation of the graft. In other preferred embodiments: a first administration of stem cells is provided prior to or at the time of implantation of a graft and a second administration of stem cells is provided subsequent to the implantation of a graft. The period between administrations of hematopoietic stem cells can be varied. In preferred embodiments a subsequent administration of hematopoietic stem cell is provided: at least two days, one week, one month, or six months after the previous administration of stem cells; at least two days, one week, one month, or six months after the implantation of the graft.

The method can further include the step of administering a second or subsequent dose of hematopoietic stem cells: when the recipient begins to show signs of rejection, e.g., as evidenced by a decline in function of the grafted organ, by a change in the host donor specific antibody response, or by a change in the host lymphocyte response to donor antigen; when the level of chimerism decreases; or generally, as is needed to maintain tolerance or otherwise prolong the acceptance of a graft. Thus, method of the invention can be modified to include a further step of determining if a subject which has received a one or more administrations of hematopoietic stem cells is in need of a subsequent administration of hematopoietic stem cells, and if so, administering a subsequent dose of hematopoietic stem cells to the recipient.

Any of the methods referred to herein can include the administration of agents, e.g., 15-deoxyspergualin, mycophenolate mofetil, brequinar sodium, or similar agents, which inactivate, e.g., inhibit the production, levels, or activity of antibodies in the recipient. One or more of these agents can be administered: prior to the implantation of donor tissue, e.g., one, two, or three days, or one, two, or three weeks before implantation of donor tissue; at the time of implantation of donor tissue; or after implantation of donor tissue, e.g., one, two, or three days, or one, two or three weeks after, implantation of a graft.

The administration of the agent can be initiated: when the recipient begins to show signs of rejection, e.g., as evidenced by a decline in function of the grafted organ, by a change in the host donor specific antibody response, or by a change in the host lymphocyte response to donor antigen; when the level of chimerism decreases; when the level of chimerism falls below a predetermined value; or generally, as is needed to maintain tolerance or otherwise prolong the acceptance of a graft.

The period over which the agent is administered (or the period over which clinically effective levels are maintained in the subject) can be long term, e.g., for six months or more or a year or more, or short term, e.g., for less than a year, more preferably six months or less, more preferably one month or less, and more preferably two weeks or less. The period will generally be at least about one week and preferably at least about two weeks in duration. In preferred embodiments the period is two or three weeks long.

Preferred embodiments include administration of 15-deoxyspergualin (6 mg/kg/day) for about two weeks beginning on the day of graft implantation.

An anti-CD2 antibody, preferably a monoclonal, e.g., BTI-322, or a monoclonal directed at a similar or overlapping epitope, can be used in addition to or in place of any anti-T cell antibodies (e.g., ATG) in any method referred to herein.

In another aspect, the invention features, a genetically engineered swine cell, e.g., a cultured swine cell, a retrovirally transformed swine cell, or a cell derived from a transgenic swine. The cell includes a transgene which encodes an intracellular antibody which binds to an α(1,3)galactosyltransferase, e.g., β-D-galactosyl-1,4-N-acetyl-D-glucosaminide α(1,3)galactosyltransferase, and inhibits the ability of the enzyme to form a galactosyl α(1,3) galactose moiety on the swine cell. The swine cell can be from a full-size swine or from a miniature swine. Preferably, the transgene is integrated into the genome of the cell.

In preferred embodiments the transgene encodes: an antibody which is targeted to the endoplasmic reticulum; a single chain antibody, e.g., a single chain variable-region fragment. (A single chain variable region fragment antibody includes immunoglobulin heavy and light chain variable region (V_{H} and V_{L}) domains joined by a flexible peptide linker.)

In preferred embodiments the genetically engineered swine cell is: a swine hematopoietic stem cell, e.g., a cord blood hematopoietic stem cell, a bone marrow hematopoietic stem cell, or a fetal or neonatal liver or spleen hematopoietic stem cell; derived from differentiated blood cells, e.g. a myeloid cell, such as a megakaryocyte, monocyte, granulocyte, or an eosinophil; an erythroid cell, such as a red blood cell, e.g. a lymphoid cell, such as B lymphocytes and T lymphocytes; derived from a pluripotent hematopoietic stem cell, e.g. a hematopoietic precursor, e.g. a burst-forming units-erythroid (BFU-E), a colony forming unit-erythroid (CFU-E), a colony forming unit-megakaryocyte (CFU-Meg), a colony forming unit-granulocyte-monocyte (CFU-GM), a colony forming unit-eosinophil, or a colony forming unit-granulocyte-erythrocyte-megakaryocyte-monocyte (CFU-GEMM); a swine cell other than a hematopoietic stem cell, or other blood cell; a swine thymic cell, e.g., a swine thymic stromal cell; a bone marrow stromal cell; a swine liver cell; a swine kidney cell; a swine epithelial cell; a swine hematopoietic progenitor cell; a swine muscle cell, e.g., a heart cell; an endothelial cell; or a dendritic cell or precursor thereof.

In yet other preferred embodiments the cell is: isolated or derived from cultured cells, e.g., a primary culture, e.g., a primary cell culture of hematopoietic stem cells; isolated or derived from a transgenic animal.

In another aspect, the invention features, a transgenic swine having cells which include a transgene which encodes an intracellular antibody which binds to an α(1,3)galactosyltransferase, e.g., β-D-galactosyl-1,4-N-acetyl-D-glucosaminide α(1,3)galactosyltransferase. The transgenic antibody inhibits the ability of the enzyme to form a galactosyl α(1,3) galactose moiety on cells of the transgenic swine. The transgenic swine can be a full-size swine or a miniature swine. Preferably, the transgene is integrated into the genome of the animal.

In preferred embodiments the transgene encodes: an antibody which is targeted to the endoplasmic reticulum; a single chain antibody, e.g., a single chain variable-region fragment. Transgenic swine (or swine cells) of the invention can be used as a source for tissue for grafting into a human recipient, e.g., hematopoietic cells or other tissues or organs.

In another aspect, the invention features, a swine organ or a swine tissue, having cells which include a transgene which encodes an intracellular antibody which binds to an α(1,3)galactosyltransferase, e.g., β-D-galactosyl-1,4-N-acetyl-D-glucosaminide α(1,3)galactosyltransferase. The transgenic antibody inhibits the ability of the enzyme to form a galactosyl α(1,3) galactose moiety on cells of the transgenic swine. The transgenic swine organ or tissue can be a full-size swine or a miniature swine organ or tissue. Preferably, the transgene is integrated into the genome of the cells.

In preferred embodiments the transgene encodes: an antibody which is targeted to the endoplasmic reticulum; a single chain antibody, e.g., a single chain variable-region fragment.

In preferred embodiments the organ is a heart, lung, kidney, pancreas, or liver.

In preferred embodiments the tissue is: thymic tissue; islet cells or islets; stem cells; bone marrow; endothelial cells; skin; or vascular tissue.

The swine organs and tissues of the invention can be used as a source of tissue for grafting into a human recipient, e.g., hematopoietic cells or other tissues or organs.

Graft tissue which expresses a transgenic anti-α(1,3)galactosyltransferase intracellular antibody can be used to improve methods of transplanting xenogeneic tissue into a recipient. For example, acceptance of swine, e.g., miniature swine or full-size swine, tissue by a human recipient can be prolonged if the porcine tissue expresses an antibody, preferably an intracellular antibody, which binds to an α(1,3)galactosyltransferase, e.g., β-D-galactosyl-1,4-N-acetyl-D-glucosaminide α(1,3)galactosyltransferase, and thereby reduces the number of galactosyl α(1,3) galactose moieties on the surfaces of a graft. Transgenic tissue described herein can be used in place of other swine tissue in any of the methods described or referred to herein.

Genetically engineered swine cells of the invention can be made by methods known to those skilled in the art, e.g., by retroviral transduction of swine cells. Methods for producing transgenic swine of the invention use standard transgenic technology. These methods include, e.g., the infection of the zygote or organism by viruses including retroviruses; the infection of a tissue with viruses and then reintroducing the tissue into an animal; and the introduction of a recombinant nucleic acid molecule into an embryonic stem cell of a mammal followed by appropriate manipulation of the embryonic stem cell to produce a transgenic animal.

As used herein, the term “transgene” refers to a nucleic acid sequence (encoding, e.g., an antibody, e.g., an intracellular antibody), which is inserted by artifice into a cell. The transgene can become part of the genome of an animal which develops in whole or in part from that cell. If the transgene is integrated into the genome it results, by its insertion, in a change in the nucleic acid sequence of the genome into which it is inserted. A transgene can include one or more transcriptional regulatory sequences and any other nucleic acid sequences, such as introns, that may be necessary for a desired level or pattern of expression of a selected nucleic acid, all operably linked to the selected nucleic acid. The transgene can include an enhancer sequence. The transgene is typically introduced into the animal, or an ancestor of the animal, at a prenatal, e.g., an embryonic, or earlier, stage. The transgene can include a sequence which targets the transgene product to the enoplasmic reticulum.

As used herein, the term “transgenic cell” refers to a cell containing a transgene.

As used herein, a “transgenic animal” is any animal in which one or more, and preferably essentially all, of the cells of the animal includes a transgene. The transgene is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

As used herein, the term “recombinant swine cells” refers to cells derived from swine, preferably miniature swine, which have been used as recipients for a recombinant vector or other transfer nucleic acid, and include the progeny of the original cell which has been transfected or transformed. Recombinant swine cells include cells in which transgenes or other nucleic acid vectors have been incorporated into the host cell's genome, as well as cells harboring expression vectors which remain autonomous from the host cell's genome.

The term “tissue” as used herein, means any biological material that is capable of being transplanted and includes organs (especially the internal vital organs such as the heart, lung, liver, kidney, pancreas and thyroid), cornea, skin, blood vessels and other connective tissue, cells including blood and hematopoietic cells, Islets of Langerhans, brain cells and cells from endocrine and other organs and bodily fluids, all of which may be candidate for transplantation.

Production of Intrabodies: Single Chain Variable Region Fragment Antibodies

Single chain variable region fragment antibodies are particularly preferred for use in methods described herein. The first step in the production of intrabodies of the invention is the production of monoclonal antibodies specific for the α(1,3)galactosyltransferase, e.g., β-D-galactosyl-1,4-N-acetyl-D-glucosaminide α(1,3)galactosyltransferase. These antibodies can be prepared by injection of the enzyme, preferably the swine enzyme, into an animal, e.g., a mouse. Antibodies produced by individual hybridomas can be tested, in vitro, for the ability to bind an block α(1,3)galactosyltransferase activity. The immunoglobulin heavy and light chain variable region (V_{H} and V_{L}) domains from an antibody which inhibits activity are cloned and used to prepare a single chain antibody construct. A construct can be evaluated for in vivo activity by transfecting it into a swine cell line and determining the effect of the antibody on presentation of the galactosyl α(1,3) galactose moiety. A construct which reduces presentation of the moiety can be used to construct a transgenic animal or to prepare genetically engineered cells.

Genetically Engineered Swine Cells

Transgenic swine cells of the invention can be produced by any methods known to those in the art. Transgenes can be introduced into cells, e.g., stem cells, e.g., cultured stem cells, by any methods which allows expression of these genes at a level and for a period sufficient to promote engraftment or maintenance of the cells. These methods include e.g., transfection, electroporation, particle gun bombardment, and transduction by viral vectors, e.g., by retroviruses. Transgenic swine cells can also be derived from transgenic animals. Recombinant retroviruses are a preferred delivery system.

Preparation of Transgenic Swine

Microinjection of Swine Oocytes

In preferred embodiments the transgenic swine of the present invention is produced by:

i) microinjecting a recombinant nucleic acid molecule into a fertilized swine egg to produce a genetically altered swine egg;

ii) implanting the genetically altered swine egg into a host female swine;

iii) maintaining the host female for a time period equal to a substantial portion of the gestation period of said swine fetus.

iv) harvesting a transgenic swine having at least one swine cell that has developed from the genetically altered mammalian egg, which expresses a human class I gene.

In general, the use of microinjection protocols in transgenic animal production is typically divided into four main phases: (a) preparation of the animals; (b) recovery and maintenance in vitro of one or two-celled embryos; (c) microinjection of the embryos and (d) reimplantation of embryos into recipient females. The methods used for producing transgenic livestock, particularly swine, do not differ in principle from those used to produce transgenic mice. Compare, for example, Gordon et al. (1983) Methods in Enzymology 101:411, and Gordon et al. (1980) PNAS 77:7380 concerning, generally, transgenic mice with Hammer et al. (1985) Nature 315:680, Hammer et al. (1986) J Anim Sci 63:269-278, Wall et al. (1985) Biol Reprod. 32:645-651, Pursel et al. (1989) Science 244:1281-1288, Vize et al. (1988) J Cell Science 90:295-300, Muller et al. (1992) Gene 121:263-270, and Velander et al (1992) PNAS 89:12003-12007, each of which teach techniques for generating transgenic swine. See also, PCT Publication WO 90/03432, and PCT Publication WO 92/22646 and references cited therein.

One step of the preparatory phase comprises synchronizing the estrus cycle of at least the donor females, and inducing superovulation in the donor females prior to mating. Superovulation typically involves administering drugs at an appropriate stage of the estrus cycle to stimulate follicular development, followed by treatment with drugs to synchronize estrus and initiate ovulation. As described in the example below, pregnant mare's serum is typically used to mimic the follicle-stimulating hormone (FSH) in combination with human chorionic gonadotropin (hCG) to mimic luteinizing hormone (LH). The efficient induction of superovulation in swine depend, as is well known, on several variables including the age and weight of the females, and the dose and timing of the gonadotropin administration. See for example, Wall et al. (1985) Biol. Reprod. 32:645 describing superovulation of pigs. Superovulation increases the likelihood that a large number of healthy embryos will be available after mating, and further allows the practitioner to control the timing of experiments.

After mating, one or two-cell fertilized eggs from the superovulated females are harvested for microinjection. A variety of protocols useful in collecting eggs from pigs are known. For example, in one approach, oviducts of fertilized superovulated females can be surgically removed and isolated in a buffer solution/culture medium, and fertilized eggs expressed from the isolated oviductal tissues. See, Gordon et al. (1980) PNAS 77:7380; and Gordon et al. (1983) Methods in Enzymology 101:411. Alternatively, the oviducts can be cannulated and the fertilized eggs can be surgically collected from anesthetized animals by flushing with buffer solution/culture medium, thereby eliminating the need to sacrifice the animal. See Hammer et al. (1985) Nature 315:600. The timing of the embryo harvest after mating of the superovulated females can depend on the length of the fertilization process and the time required for adequate enlargement of the pronuclei. This temporal waiting period can range from, for example, up to 48 hours for larger breeds of swine. Fertilized eggs appropriate for microinjection, such as one-cell ova containing pronuclei, or two-cell embryos, can be readily identified under a dissecting microscope.

The equipment and reagents needed for microinjection of the isolated swine embryos are similar to that used for the mouse. See, for example, Gordon et al. (1983) Methods in Enzymology 101:411; and Gordon et al. (1980) PNAS 77:7380, describing equipment and reagents for microinjecting embryos. Briefly, fertilized eggs are positioned with an egg holder (fabricated from 1 mm glass tubing), which is attached to a micro-manipulator, which is in turn coordinated with a dissecting microscope optionally fitted with differential interference contrast optics. Where visualization of pronuclei is difficult because of optically dense cytoplasmic material, such as is generally the case with swine embryos, centrifugation of the embryos can be carried out without compromising embryo viability. Wall et al. (1985) Biol. Reprod. 32:645. Centrifugation will usually be necessary in this method. A recombinant nucleic acid molecule of the present invention is provided, typically in linearized form, by linearizing the recombinant nucleic acid molecule with at least 1 restriction endonuclease, with an end goal being removal of any prokaryotic sequences as well as any unnecessary flanking sequences. In addition, the recombinant nucleic acid molecule containing the tissue specific promoter and the human class I gene may be isolated from the vector sequences using 1 or more restriction endonucleases. Techniques for manipulating and linearizing recombinant nucleic acid molecules are well known and include the techniques described in Molecular Cloning: A Laboratory Manual, Second Edition. Maniatis et al. eds., Cold Spring Harbor, N.Y. (1989).

The linearized recombinant nucleic acid molecule may be microinjected into the swine egg to produce a genetically altered mammalian egg using well known techniques. Typically, the linearized nucleic acid molecule is microinjected directly into the pronuclei of the fertilized eggs as has been described by Gordon et al. (1980) PNAS 77:7380-7384. This leads to the stable chromosomal integration of the recombinant nucleic acid molecule in a significant population of the surviving embryos. See for example, Brinster et al. (1985) PNAS 82:4438-4442 and Hammer et al. (1985) Nature 315:600-603. The microneedles used for injection, like the egg holder, can also be pulled from glass tubing. The tip of a microneedle is allowed to fill with plasmid suspension by capillary action. By microscopic visualization, the microneedle is then inserted into the pronucleus of a cell held by the egg holder, and plasmid suspension injected into the pronucleus. If injection is successful, the pronucleus will generally swell noticeably. The microneedle is then withdrawn, and cells which survive the microinjection (e.g. those which do not lysed) are subsequently used for implantation in a host female.

The genetically altered mammalian embryo is then transferred to the oviduct or uterine horns of the recipient. Microinjected embryos are collected in the implantation pipette, the pipette inserted into the surgically exposed oviduct of a recipient female, and the microinjected eggs expelled into the oviduct. After withdrawal of the implantation pipette, any surgical incision can be closed, and the embryos allowed to continue gestation in the foster mother. See, for example, Gordon et al. (1983) Methods in Enzymology 101:411; Gordon et al. (1980) PNAS 77:7390; Hammer et al. (1985) Nature 315:600; and Wall et al. (1985) Biol. Reprod. 32:645.

The host female mammals containing the implanted genetically altered mammalian eggs are maintained for a sufficient time period to give birth to a transgenic mammal having at least 1 cell, e.g. a bone marrow cell, e.g. a hematopoietic cell, which expresses the recombinant nucleic acid molecule of the present invention that has developed from the genetically altered mammalian egg.

At two-four weeks of age (post-natal), tail sections are taken from the piglets and digested with Proteinase K. DNA from the samples is phenol-chloroform extracted, then digested with various restriction enzymes. The DNA digests are electrophoresed on a Tris-borate gel, blotted on nitrocellulose, and hybridized with a probe consisting of the at least a portion of the coding region of the recombinant cDNA of interest which had been labeled by extension of random hexamers. Under conditions of high stringency, this probe should not hybridize with the endogenous pig gene, and will allow the identification of transgenic pigs.

The methods of the invention can also include inducing tolerance to the galactosyl α(1, 3) galactose moiety by administering to the recipient blood group B antigen. This can be done prior to exposure to the galactosyl α(1, 3) galactose moiety tolerogen, so as to induce a first level of tolerance to the galactosyl α(1, 3) galactose moiety.

Equivalents

Those skilled in the art will recognize, or be able to ascertain using no more than routine experimentation, many equivalents of the specific embodiments of the invention described herein. Such equivalents are intended to be encompassed by the following claims.

Other embodiments are within the following claims.

What is claimed is:

1. A method of promoting, in a recipient mammal of a first species which does not possess UDP galactose:β-D-galactosyl-1,4-N-acetyl-D-glucosaminide α(1,3)galactosyltransferase (α1,3GT) activity or which does not present galactosyl α(1, 3) galactose moieties on its cells, tolerance to the galactosyl α(1, 3) galactose moiety, or to a graft which produces or displays the galactosyl α(1, 3) galactose moiety, comprising:
providing to the recipient mammal a tolerance-inducing galactosyl α(1, 3) galactose moiety,
thereby inducing tolerance to the galactosyl α(1, 3) galactose moiety or to a graft which presents the galactosyl α(1, 3) galactose moiety.

2. The method of claim 1, wherein the galactosyl α(1, 3) galactose moiety is presented on a modified cell of the recipient.

3. The method of claim 1, further comprising implanting a graft in the recipient.

4. The method of claim 1, wherein said recipient is a human and said graft is from a swine.

5. The method of claim 4, wherein said swine is a miniature swine.

6. The method of claim 1, further comprising inactivating galactosyl α(1, 3) galactose moiety reactive antibodies.

7. The method of claim 1, further comprising administering swine hematopoietic stem cells to said recipient and wherein said graft and said hematopoietic stem cells are taken from the same donor animal.

8. The method of claim 1, further comprising administering swine hematopoietic stem cells to said recipient and wherein said graft and said hematopoietic stem cells are taken from animals from an inbred herd of miniature swine.

9. A method of promoting, in a recipient mammal of a first species which does not possess UDP galactose:β-D-galactosyl-1,4-N-acetyl-D-glucosaminide a (1,3)galactosyltransferase (α1,3GT) activity or which does not present galactosyl α(1, 3) galactose moieties on its cells, tolerance to the galactosyl α(1, 3) galactose moiety, or to a graft which presents the galactosyl α(1, 3) galactose moiety, comprising:
providing a cell from the recipient mammal which has been modified so as to produce or display the galactosyl α(1, 3) galactose moiety, thereby inducing tolerance to the galactosyl α(1, 3) galactose moiety or to a graft which presents the galactosyl α(1, 3) galactose moiety.

10. The method of claim 9, further comprising implanting said graft in said recipient.

11. The method of claim 9, wherein said recipient is a human and said graft is from a swine.

12. The method of claim 9, wherein said swine is a miniature swine.

13. The method of claim 9, further comprising inactivating galactosyl α(1, 3) galactose moiety reactive antibodies.

14. The method of claim 9, further comprising administering swine hematopoietic stem cells to said recipient and wherein said graft and said hematopoietic stem cells are taken from the same donor animal.

15. The method of claim 9, further comprising administering swine hematopoietic stem cells to said recipient and wherein said graft and said hematopoietic stem cells are taken from animals from an inbred herd of miniature swine.

16. A method of promoting, in a recipient mammal of a first species which does not possess UDP galactose:β-D-galactosyl-1,4-N-acetyl-D-glucosaminide α(1,3)galactosyltransferase (α1,3GT) activity or which does not present galactosyl α(1, 3) galactose moieties on its cells, tolerance to the galactosyl α(1, 3) galactose moiety or to a graft which produces or displays the galactosyl α(1, 3) galactose moiety, comprising:
providing a cell from the recipient mammal, into which cell has been inserted a nucleic acid encoding a protein which promotes the formation of the galactosyl α(1, 3) galactose moiety,
thereby inducing tolerance to the galactosyl α(1, 3) galactose moiety or to a graft which presents the galactosyl α(1, 3) galactose moiety.

17. The method of claim 16, further comprising introducing a graft into said recipient.

18. The method of claim 17, wherein said recipient is a human and said graft is from a swine.

19. The method of claim 18, wherein said swine is a miniature swine.

20. The method of claim 16, wherein said nucleic acid encodes a protein which promotes the addition of a terminal galactosyl residue to a galactosyl residue.

21. The method of claim 21, wherein the nucleic acid encodes an α(1,3)galactosyltransferase.

22. The method of claim 17, wherein said graft includes a kidney.

23. The method of claim 16, wherein said cell is a hematopoietic stem cell.

24. The method of claim 16, further comprising inactivating galactosyl α(1, 3) galactose moiety reactive antibodies.

25. The method of claim 17, further comprising administering swine hematopoietic stem cells to said recipient and wherein said graft and said hematopoietic stem cells are taken from the same donor animal.

26. The method of claim 17, further comprising administering swine hematopoietic stem cells to said recipient and wherein said graft and said hematopoietic stem cells are taken from animals from an inbred herd of miniatures swine.

27. A method of promoting, in a recipient mammal of a first species which does not possess UDP galactose:β-D-galactosyl-1,4-N-acetyl-D-glucosaminide α(1,3)galactosyltransferase (α1,3GT) activity or which does not present galactosyl α(1, 3) galactose moieties on its cells, tolerance to the galactosyl α(1, 3) galactose moiety or to a graft which presents the galactosyl α(1, 3) galactose moiety comprising:
forming a galactosyl α(1, 3) galactose moiety on the surface of a cell of the recipient mammal,
thereby inducing tolerance to the galactosyl α(1, 3) galactose moiety.

28. The method of claim 27, further comprising introducing the graft into the recipient.

29. The method of claim 28, wherein said recipient is a human and said graft is from a swine.

30. The method of claim 29, wherein said swine is a miniature swine.

31. The method of claim 27, wherein said graft includes a kidney, liver, and heart.

32. The method of claim 27, wherein said cell is a hematopoietic stem cell.

33. The method of claim 27, wherein the galactosyl α(1,3) galactose moiety is formed by contacting the cell with a protein which results in the formation of a galactosyl α(1,3) galactose moiety on the surface of the cell.

34. The method of claim 33, wherein, the cell is contacted with a α(1,3)galactosyltransferase.

35. The method of claim 27, further comprising inactivating galactosyl α(1, 3) galactose moiety reactive antibodies.

36. The method of claim 27, further comprising administering swine hematopoietic stem cells to said recipient and wherein said graft and said hematopoietic stem cells are taken from the same donor animal.

37. The method of claim 27, further comprising administering swine hematopoietic stem cells to said recipient and wherein said graft and said hematopoietic stem cells are taken from animals from an inbred herd of miniature swine.

38. A method of inactivating a recipient natural antibody against a galactosyl α(1, 3) galactose moiety on a graft by administering to said recipient anti-idiotypic antibodies, or fragments thereof, against the natural antibody.

39. The method of claim 38, further comprising implanting the graft in the recipient.

40. The method of claim 39, wherein the recipient is a human and the galactosyl α(1, 3) galactose moiety is on a swine graft.

41. The method of claim 40, wherein said swine graft is a miniature swine graft.

42. The method of claim 38, further comprising administering swine hematopoietic stem cells to said recipient and wherein said graft and said hematopoietic stem cells are taken from the same donor animal.

43. The method of claim 38, further comprising administering swine hematopoietic stem cells to said recipient and wherein said graft and said hematopoietic stem cells are taken from animals from an inbred herd of miniature swine.

44. A method of promoting tolerance to carbohydrate antigen in a recipient human which recipient does not produce or display the antigen on or in its cells, tissues, or organs comprising:
providing to the recipient a recipient cell which produces or displays tolerance-inducing antigen thereby inducing tolerance to the antigen or to a graft which produces or displays the antigen.

45. A method of promoting, in a recipient mammal tolerance to a carbohydrate antigen moiety from a donor mammal of the same species, wherein the antigen is not expressed in the recipient but is expressed in the donor, comprising
providing to the recipient mammal a recipient cell which produces or displays tolerance-inducing a carbohydrate antigen moiety.

46. The method of claim 45, wherein said antigen is a blood group carbohydrate.

47. The method of claim 45, wherein said antigen is a blood group A carbohydrate moiety.

48. The method of claim 45, wherein said antigen is a blood group B carbohydrate moiety.

49. The method of claim 45, wherein said antigen is a blood group H carbohydrate moiety.

50. The method of claim 45, wherein said antigen is a blood group Le carbohydrate moiety.

51. The method of claim 45, wherein said antigen is a human and the antigen is a blood group I carbohydrate moiety.

52. The method of claim 45, wherein said recipient cell is modified to express UDP-GalNAc:Fucα1,2Gal-Rα1,3-GalNAc transferase (EC 2.4.1.40), or an enzyme of equivalent activity.

53. The method of claim 45, wherein said recipient cell is modified to express UDP-GalNAc:Fucα1,2Gal-Rα1,3Gal transferase (EC 2.4.1.37), or an enzyme of equivalent activity.

54. The method of claim 45, wherein said recipient cell is modified to express GDP-Fuc:βgalactosideα2-Fuc-transferase (EC 2.4.1.69), or an enzyme of equivalent activity.

55. The method of claim 45, wherein said recipient cell is modified to express GDP-Fuc:Galβ1,3/4GlcNAc-Rα4/3Fuc transferase (EC 2.4.1.65), or an enzyme of equivalent activity.

56. The method of claim 45, wherein said recipient cell is modified to express UDP-GlcNAc:GlcNAcβ1,3Galβ1,4GlcNAc-Rβ6-GlcNAc transferase, or an enzyme of equivalent activity.

57. The method of claim 45, wherein said method further includes introducing a graft from said donor mammal into said recipient mammal.
